SEQUENCE LISTING (39 sequences)

SEQ ID NO: 1 - DNA, 570 bp, Mus musculus
gacggcatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagac

SEQ ID NO: 2 - DNA, 207 bp, Neurospora crassa
atggcctccactcgtgtcctcgcctctcgcctggcctcccagatggctgcttccgccaaggttgcccgccctgctgtccgcgttgctcaggtcagcaagcgcaccatccagactggctcccccctccagaccctcaagcgcacccagatgacctccatcgtcaacgccaccacccgccaggctttccagaagcgcgcctactcttcc

SEQ ID NO: 3 - protein, 259 aa, Artificial
  Fusion protein pSu9-DHFR
MASTRVLASRLASQMAASAKVARPAVRVAQVSKRTIQTGSPLQTLKRTQMTSIVNATTRQAFQKRAYSSDGIMVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 4 - DNA, 76 bp, Arabidopsis thaliana
ggggatgtagctcatatggtagagcgctcgctttgcatgcgagaggcacagggttcgattccctgcatctccacca

SEQ ID NO: 5 - DNA, 717 bp, Aequorea victoria
atgggtgaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctgttatggtgtacaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggcagacaaacaaaagaatggaaccaaagttaacttcaaaattagacgcaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 6 - DNA, 263 bp, Artificial
  chimera pBluescript / mitochondrial tRNA(His) of larch
gcgaattggagctccaccgcggtggcggcgctctagaactagtggatccaatagcgagtatagacgtgtctaagggcgagcataacccaataggtcagagtgtcagattgtgaattcgaaaacacgggttcgaatcccgttattcgccgcaccccattgtcgaccaggccctctcccccctattaaataagtaaagcgatggccccggctttcaatacgttacaatctatttattcccctaaaggatccccccgggctgcagg

SEQ ID NO: 7 - DNA, 22 bp, Artificial
  primer
taggggggagagggcctcctcg

SEQ ID NO: 8 - DNA, 21 bp, Artificial
  primer
gcgagtatagacgtgtctaag

SEQ ID NO: 9 - DNA, 777 bp, Artificial
  precursor form of larch mitochondrial tRNAHis fused with mitochondrial Atp9 of potato
cttatgagttcgatacgtaggttgcgcaaccctcgagagggtataccagctggacgtccgccggcgcttaagtgatcactagcgagtatagacgtgtctaagggcgagcataacccaataggtcagagtgtcagattgtgaattcgaaaacacgggttcgaatcccgttattcgccgcaccccattgtcgaccaggccctctcccctagcctatgctttgcatgaacatctcaatgtccaagataaaaagaacgaggggaagaatcgacgaggcgagtgttctcgaagagaaaatcgtgatggaaaaagcgtgaggagaattcgaaactcgagatgttagaaggtgcaaaatyaatgggtgcaggagctgctacaattgcttyagcgggagctgctatcggtattggaaacgtyyttagttcytygattcattccgtggcgcgaaatccatcattggcaaaacaattatttggttatgccattttgggctttgctctaaccgaagctattgcatygtttgcccyaatgatggcctttttgatctyattcgtattcyaagttcgttagtaatcgtttacggtgggtggataagcaggaagggatccctgtggttagactaactggccgagaaggctagtgaggttcctgctatggtgaagtgaaagatctttcactatagtgggaagaagacaggtgggagcgagccgagcgagagcaaagcaagtttcagtggtgggctgtcttcgcggtcccattagcttaagggcgccggcggta

SEQ ID NO: 10 - DNA, 653 bp, Artificial
  Mitochondrial Atp9 of potato
cttatgagttcgatacgtaggttgcgcaaccctcgagagggtataccagctggacgtccgccggcgcttaagtgatcactactagcctatgctttgcatgaacatctcaatgtccaagataaaaagaacgaggggaagaatcgacgaggcgagtgttctcgaagagaaaatcgtgatggaaaaagcgtgaggagaattcgaaactcgagatgttagaaggtgcaaaatyaatgggtgcaggagctgctacaattgcttyagcgggagctgctatcggtattggaaacgtyyttagttcytygattcattccgtggcgcgaaatccatcattggcaaaacaattatttggttatgccattttgggctttgctctaaccgaagctattgcatygtttgcccyaatgatggcctttttgatctyattcgtattcyaagttcgttagtaatcgtttacggtgggtggataagcaggaagggatccctgtggttagactaactggccgagaaggctagtgaggttcctgctatggtgaagtgaaagatctttcactatagtgggaagaagacaggtgggagcgagccgagcgagagcaaagcaagtttcagtggtgggctgtcttcgcggtcccattagcttaagggcgccggcggta

SEQ ID NO: 11 - DNA, 41 bp, Artificial
  oligonucleotide
tcagaccaggccctctcccctagcctatgctttgcatgaac

SEQ ID NO: 12 - DNA, 22 bp, Artificial
  oligonucleotide
aatgggaccgcgaagacagccc

SEQ ID NO: 13 - DNA, 21 bp, Artificial
  oligonucleotide
gcgagtatagacgtgtctaag

SEQ ID NO: 14 - DNA, 28 bp, Artificial
  oligonucleotide
caaagcataggctaggggagagggcctg

SEQ ID NO: 15 - DNA, 19 bp, Artificial
  primer
cgcgttgggagctctccca

SEQ ID NO: 16 - DNA, 19 bp, Artificial
  primer
gcggccgcgggaattcgat

SEQ ID NO: 17 - DNA, 239 bp, Artificial
  precursor form of larch mitochondrial tRNAHis fused on side 3 with the sequence coding for the stem-loop portion of the MS2 RNA
gcgagtatagacgtgtctaagggcgagcataacccaataggtcagagtgtcagattgtgaattcgaaaacacgggttcgaatcccgttattcgccgcaccccattgtcgaccaggccctctcccggatcctaaggtacctaattgcctagaaaacatgaggatcacccatgtctgcaggtcgactctagaaaacatgaggatcacccatgtctgcagtattcccgggttcattagatcc

SEQ ID NO: 18 - DNA, 564 bp, Artificial
  Mitochondrial targeting sequence pSu9 fused with the CP protein
atggcctccactcgtgtcctcgcctctcgcctggcctcccagatggctgcttccgccaaggttgcccgccctgctgtccgcgttgctcaggtcagcaagcgcaccatccagactggctcccccctccagaccctcaagcgcacccagatgacctccatcgtcaacgccaccacccgccaggctttccagaagcgcgcctactcttccatggcttctaactttactcagttcgttctcgtcgacaatggcggaactggagacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggat

SEQ ID NO: 19 - protein, 188 aa, Artificial
  Synthetic Construct
MASTRVLASRLASQMAASAKVARPAVRVAQVSKRTIQTGSPLQTLKRTQMTSIVNATTRQAFQKRAYSSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAD

SEQ ID NO: 20 - DNA, 21 bp, Artificial
  primer
gcgagtatagacgtgtctaag

SEQ ID NO: 21 - DNA, 25 bp, Artificial
  primer
ccttaggatccgggagagggcctgg

SEQ ID NO: 22 - DNA, 25 bp, Artificial
  primer
ccaggccctctcccggatcctaagg

SEQ ID NO: 23 - DNA, 21 bp, Artificial
  primer
cgcgcgcagatctaatgaacc

SEQ ID NO: 24 - DNA, 19 bp, Artificial
  primer
atggcctccactcgtgtcc

SEQ ID NO: 25 - DNA, 23 bp, Artificial
  primer
gttagaagccctggaagagtagg

SEQ ID NO: 26 - DNA, 23 bp, Artificial
  primer
cctactcttccctggcttctaac

SEQ ID NO: 27 - DNA, 23 bp, Artificial
  primer
atggatccgcgtagatgccggag

SEQ ID NO: 28 - DNA, 28 bp, Artificial
  tRNAHis 5′ end
gcgagtatagacgtgtctaaggcgagca

SEQ ID NO: 29 - DNA, 16 bp, Artificial
  tRNAHis 3′ end
gttattcgccgcaccc

SEQ ID NO: 30 - DNA, 15 bp, Artificial
  tRNAHis 3′ end
gttattcgccgcacc

SEQ ID NO: 31 - DNA, 13 bp, Artificial
  tRNAHis 3′ end
gttattcgccgca

SEQ ID NO: 32 - DNA, 12 bp, Artificial
  tRNAHis 3′ end
gttattcgccgc

SEQ ID NO: 33 - DNA, 11 bp, Artificial
  tRNAHis 3′ end
gttattcgccg

SEQ ID NO: 34 - DNA, 13 bp, Artificial
  tRNAHis 3′ end
gttattcgcccca

SEQ ID NO: 35 - DNA, 133 bp, Artificial
  NUcleic acid of interest anticox-2SL
ataatgttatataagtaataatataataaaatatcctaaggtacctaattgcctagaaaacatgaggatcacccatgtctgcaggtcgactctagaaaacatgaggatcacccatgtctgcagtattcccggg

SEQ ID NO: 36 - DNA, 45 bp, Artificial
  primer
ataatgttatataagtaataatataataaaatatcctaaggtacc

SEQ ID NO: 37 - DNA, 23 bp, Artificial
  primer
cccgggaatactgcagacatggg

SEQ ID NO: 38 - DNA, 19 bp, Artificial
  oligonucleotidic probe
gtgagaatagctggtgttg

SEQ ID NO: 39 - DNA, 20 bp, Artificial
  oligonucleotidic probe
ggctcctcatagggggctcg

(19) United States
(12) Patent Application Publication
(10) Pub. No.: US 2012/0110693 A1
(43) Pub. Date: May 3, 2012

(54) TARGETING NUCLEIC ACIDS IN MITOCHONDRIA

(76) Inventors: Laurence Drouard, Niederhausbergen (FR); Antonio Placido, Castellaneta (IT); Francois Sieber, Lampertheim (FR)

(21) Appl. No.: 13/266,431

(22) PCT Filed: Apr. 27, 2010
(86) PCT No.: PCT/FR2010/050792

§ 371 Date: Jan. 12, 2012

(30) Foreign Application Priority Data
Apr. 27, 2009 (FR) 0952740

(51) Int. Cl. A01H 1/06; C07K 19/00; C12N 15/62; C12N 15/79; A01H 5/10; C12N 15/85; C12N 15/81; C12N 15/82; C12N 15/80; A01H 5/00; C12N 1/00; A61K 38/02

(52) U.S. Cl. 800/278; 435/317.1; 530/350; 536/23.4; 435/320.1; 514/1.1; 435/455; 435/468; 435/471; 800/298

(57) ABSTRACT

The present invention relates to a shuttle system with which nucleic acids of interest may be imported into a mitochondrion. This system is based on the use of a fusion protein between a mitochondrial targeting sequence and a protein binding a nucleic acid of interest. This shuttle system is for example useful in agronomics, in the field of gene therapy, and within the scope of research products aiming at characterizing the function of mitochondrial genes.

The present invention relates to a shuttle system for importing nucleic acids of interest into a mitochondrion. This system is based on the use of a fusion protein between a mitochondrial targeting sequence and a protein binding a nucleic acid of interest. This shuttle system is for example useful in the field of agronomics, in the field of gene therapy, and within the scope of research projects aiming at characterizing the function of mitochondrial genes.

Mitochondria are organites present in the quasi-totality of eukaryotic cells. They are involved in many fundamental processes such as the production of ATP by oxidative phosphorylation, the synthesis of amino acids and programmed cell death. It is recognized today that the mitochondrion stems from the endosymbiosis of an α-proteobacterium inside a proto-eukaryotic cell. Consequently, mitochondria have their own genetic material but the latter is only the remnant of that of the ancestral α-proteobacterium. It only still codes for a limited number of genes and the large majority of macromolecules (e.g. about 1,500 proteins and a few RNAs) required for mitochondrial biogenesis today depend on the expression of nuclear genes. These macromolecules are then transported from the cytosol to the organelle.

Accordingly, at a very early stage in evolution, this organite had to develop transport systems which allow these macromolecules to cross the double mitochondrial membrane. This is most particularly true for proteins and the major components of the mechanisms for importing these macromolecules have been identified in all eukaryotes. In particular, the TOM (Translocase of the Outer Mitochondrial membrane) complex represents the main door for the entry of matrix proteins at the mitochondrial external membrane and the TIM (Translocase of the Inner Mitochondrial membrane) complex is required for translocation through the internal membrane (Dolezal et al., Science, 313, 314-318, 2006; Bolender et al., EMBO Rep, 9, 42-49, 2008). Further, the transport mechanism of the proteins requires a particular sequence, the mitochondrial targeting sequence, generally located at the N-terminal end of the protein and recognized by the TOM complex (Habib S J, Methods Cell Biol, 80, 761-781, 2007). Once the protein is translocated into the matrix space, this targeting sequence is then cleaved by a peptidase. Stan et al., (Mol Cell Biol, 23, 2239-2250, 2003) thus describe that the DHFR protein fused with the mitochondrial targeting sequence pSu9 is imported into yeast mitochondria.

Comparatively to proteins, a more restricted number of RNAs is imported into the mitochondria. Nevertheless, this RNA transport is also essential to cell viability. These are non-coding RNAs and more particularly transfer RNAs (tRNAs). Unlike proteins, the mechanisms for transporting RNAs into the mitochondria are poorly known and seem to resort to different protein components depending on the organisms (Salinas et al., Trends in Biochem, 33, 320-329, 2008).

The mitochondrial genome undergoes many point mutations (in particular in mammals), insertions, deletions or recombinations (for example in higher plants). These more or less significant modifications of mitochondrial DNA result in malfunctions which generate serious repercussions on the operation of the cell. Such malfunctions are at the centre of different neurodegenerative and neuromuscular human diseases, of diabetes, of ageing, and even of certain cancers (Bonnet et al., Biochem Biophys Acta, 1783, 1707-1717, 2008; Florentz et al., Cell Mol Life Sci, 60, 1356-1375, 2003; Trifunovic et al., Nature, 429, 417-423, 2004). In plants, malfunctioning of the mitochondria, often related to the presence of unusual coding sequences in the genome of the organelle, is at the origin of the cytoplasmic male sterility (CMS) phenomenon. This phenomenon is expressed by the inability of plants to produce functional pollen and is a tool highly used in the production of hybrid seeds which are more sturdy in culture like maize, cotton or rice (Budar et Pelletier, C R Acad Sci, 324, 543-550, 2001).

This organite is therefore found at the centre of many research programs but the major challenges which remain to be addressed, come up against several scientific and technical barriers. One of the most important barriers is the absence of a reliable and easy technique to be applied for transforming plant or human mitochondrial DNA in a stable way. To this day, only the transformation of the mitochondrial genome of the Saccharomyces cerevisiae yeast (Fox et al., Proc Natl Acad Sci, 85, 7288-7292, 1988) and of the Chlamydomonas reinhardtii green alga (Remacle et al., Proc Natl Acad Sci, 103, 4771-4776, 2006) was able to be obtained.

One of the possible approaches for complementing a mutated mitochondrial gene is to introduce a gene into the nuclear genome of the cell and to import the protein coded by this gene into the mitochondrion by using the mitochondrial import route for proteins. However, this technique only allows complementation of the mitochondrial malfunctions, the origin of which is proteinic. Indeed, with this approach, it is not possible to complement mitochondrial diseases for example due to mitochondrial tRNA mutations, or directly influence the replication, maintenance or expression of the genome of the mitochondrion. Further, the main limit which this method has, is to deal with difficult intracellular traffic of strongly hydrophobic proteins coded by the mitochondrial genome.

Also, it was contemplated to send messenger RNA (mRNA) to the periphery of mitochondria, in order to then target the corresponding protein in the mitochondrion. Indeed, it was demonstrated both in S. cerevisiae and in human cells, that mRNAs coding for mitochondrial proteins are enriched at the surface of the organelles. Thus, the proteins translated from these mRNAs on cytosolic polysomes located at the surface of the organelles are straightaway carried away through the double mitochondrial membrane. The significance of the 3′ UTR (3′ untranslated region) regions in the mitochondrial localization of the mRNAs has led researchers to use these sequences for targeting strongly hydrophobic proteins and thereby compensating for the mitochondrial deficiencies causing optical neuropathies (Bonnet et al., Biochem Biophys Acta, 1783, 1707-1717, 2008).

The transport of foreign nucleic acids in isolated mitochondria in a transient or stable way, and this in order to study or manipulate the expression of the mitochondrial genome, has only been successful in a limited number of cases by using the following techniques.

Vestweber et al. (Biochem Soc Tra, 17:5, 827-828, 1989) have shown that the DHFR peptide fused with the coxIV mitochondrial targeting sequence is imported into the mitochondrion. Further, this protein may carry away with it an oligonucleotide of small size when the latter is bound to the protein in a covalent way. As the oligonucleotide is covalently bound to the fusion protein, this method is not very interesting in terms of applications for allotopic expression.

The nucleic acid of interest may be imported into plant mitochondria isolated by electroporation (Farre et Araya, Nucleic Acid Res, 29, 2484-2491, 2001) or via a direct route (Koulintchenko et al., EMBO J, 22, 1245-1254, 2003). The introduced genes were then expressed under the dependency of a mitochondrial promoter. However the in vitro DNA transport in mitochondria isolated by electroporation suffers from two major problems. On the one hand, the electroporation technique causes a loss of integrity of a non-neglible portion of the organelles. On the other hand, this technique cannot be applied to entire cells for the time being. DNA transport via a direct route is not as deleterious as electroporation. On the other hand, the expression of the inserted transgene remains random, weak and difficult to obtain.

Another technique is based on the use of PNA (Peptide Nucleic Acid) molecules, i.e. macromolecules which mimic DNA. The PNA molecules are fused with a mitochondrial targeting sequence and may be imported into mitochondria of human cells (Muratovska et al., Nucleic Acids Res, 29, 1852-1863, 2001). However the PNA-peptide bond is difficult to achieve and the conjugate is vulnerable to proteases.

Another technique is based on the use of the natural RNA transport in mitochondria. In the large majority of the cases, these are tRNAs coded by the nuclear genome which are transported into the organite. This transport was experimentally demonstrated in several organisms, including humans (Salinas et al., Trends Biochem Sci, 33, 320-329, 2008; Rubio et al., Proc Natl Acad Sci, 105, 9186-9191, 2008). Recently, articles have described the possibility of importing a tRNALys into human mitochondria. Indeed, the absence of functional mitochondrial tRNALys leads to the MERRF (Myoclonic Epilepsy and Red Ragged Fibers) syndrome, which results in a serious degenerative disease. Kolesnikova et al. (Human Mol Genet, 13, 2519-2534, 2004) describe the transport of yeast tRNALys or of mutating forms, by using an RNA transport system present in human mitochondria. Mahata et al. (Science, 314, 471-474, 2006) describe the transport of human cytosolic tRNALys in mitochondria of human cells by providing a protein complex for tRNA import (called RIC for RNA Import Complex) from the Leishmania protozoan. Finally, the transport in mitochondria of human cells of small antisense RNAs under the dependency of a Leishmania signal sequence for importing tRNA and in the presence of RIC complex, has allowed specific degradation of the targeted RNAs (Mukherjee et al., Human Mol Genet, 17, 1292-1298, 2008). However, the in vivo transport of tRNALys or of its derivatives, either by using a still unknown transport system in human mitochondria, or by using the RIC complex, cannot be generalized to any tRNA. Further, the use of the RIC complex which includes about twelve sub-units and its insertion into mitochondrial membranes are difficult to apply. Finally, no large size RNA, such as for example mRNA, has been transported to this day with these systems.

These various approaches therefore each have several drawbacks which range from the instability of the molecules to the difficulty of internalization or expression. Further, they limit the fields of application because of the absence of a generalizable system. Indeed, each of the approaches mentioned above is only possible for a specific nucleic acid class.

It would therefore be desirable to obtain a tool allowing the import of nucleic acids into mitochondria which (i) would raise the present restriction as to the very small diversity of the nucleic acids which have been able to be sent into the mitochondria; (ii) would raise the present restriction as to the small size of the nucleic acids which were able to be sent into the mitochondria; (iii) would avoid the unwieldiness of the presently available techniques; and (iv) would be able to ensure the functionality and stability of the transported nucleic acids.

### DESCRIPTION OF THE INVENTION

The inventors have established a strategy for developing a protein shuttle system allowing efficient introduction “if desired” of any nucleic acid into the mitochondria. This protein shuttle system is based on the use of a protein capable of binding nucleic acids in a non-covalent way. This protein, overexpressed in fusion with a mitochondrial targeting sequence, is internalized in the mitochondrion by carrying away the allogenic nucleic acid with it.

More particularly, the inventors have identified a soluble protein capable of binding nucleic acids in an aspecific way and without resorting to a chemical reaction causing covalent bonds, mouse cytosolic DiHydroFolate Reductase (DHFR). They fused this protein with a mitochondrial targeting sequence, in this case that of the sub-unit 9 of ATP synthase (atp9) of Neurospora crassa. This fusion protein is called pSu9-DHFR.

The inventors have demonstrated the fusion protein pSu9-DHFR allows to import several different nucleic acids, i.e. plant cytosolic tRNAAla, larch mitochondrial tRNAHis precursor, and mRNA of the potato mitochondrial atp9 gene, in isolated potato mitochondria. Some of these RNAs are of large size, thus, the shuttle system according to the invention allowed import of a transcript of about 250 nucleotides corresponding to the precursor form of larch mitochondrial tRNA(His), and that of a complete mRNA of about 650 nucleotides, i.e. the mRNA of potato mitochondrial atp9 gene.

Further, it was shown that the fusion protein pSu9-DHFR allows import of RNA not only in potato mitochondria but also in yeast mitochondria. The shuttle system according to the invention may therefore be generalized to mitochondria of any organism. This system also allows import of nucleic acids other than RNAs, since it has been shown that a DNA of 75 nucleotides may be imported into potato and yeast mitochondria.

The shuttle system according to the invention therefore allows import of any type of nucleic acid, even of large size, inside mitochondria coming from any organism. Further, the procedure for applying the invention is simple and efficient. Finally, this shuttle system may be used for importing nucleic acids in a targeted and specific way, by applying it with a fusion protein capable of binding nucleic acids in a sequence-specific way.

1. Fusion Proteins According to the Invention

The invention relates to fusion proteins between a mitochondrial targeting sequence and a protein binding a nucleic acid of interest. Such fusion proteins are called <<fusion proteins according to the invention>> herein.

Any mitochondrial targeting sequence may be used within the scope of the invention. Such sequences are well known to one skilled in the art and for example include pSu9. Other mitochondrial targeting sequences are for example that of the sub-unit IV of cytochrome oxidase (coxIX) of the yeast Saccharomyces cerevisiae (Menand et al., Proc Natl Acad Sci, 95, 11014-11019, 1998), that of the sub-unit VIII of cytochrome oxidase of Schizosaccharomyces pombe (Ozawa et al., Nature Methods, 4, 413-419, 2007) or further that of the sub-unit F1β of atp synthase of Nicotiana plumbaginifolia (Moberg et al., J Mol Biol, 336, 1129-1140, 2004) This list is not exhaustive and the mitochondrial targeting sequence may be any correctly predicted mitochondrial targeting sequence and the function of which has been experimentally demonstrated, according to criteria well known to one skilled in the art (Habib et al., Methods in Cell Biology, 80, 761-781, 2007).

In a preferred embodiment, the mitochondrial targeting sequence according to the invention comprises or consists of the pSu9 sequence of the gene atp9 of Neurospora crassa. By <<pSu9 mitochondrial targeting sequence>> is meant here the polypeptide either coded by the sequence SEQ ID NO: 2, or by nucleotide sequences derived from SEQ ID NO: 2. Such derived nucleotide sequences may for example correspond to:
a fragment of at least 50, 75, 100, 125, 150, 175 or 200 consecutive nucleotides of the sequence SEQ ID NO:2;
a sequence at least 80%, 85%, 90%, 95%, 96%, 97%, 98% or 99% identical to SEQ ID NO:2;
an allelic variant of the sequence SEQ ID NO:2;
a homologous sequence to pSu9 from a species other than Neurospora crassa;
   provided that the targeting sequences coded by such derived sequences retain their capability of addressing the fusion protein to the mitochondrion. This capability may easily be checked by one skilled in the art, for example by using the procedures described in Example 1.1.7 or by Pujol et al. (Proc Natl Acad Sci, 105, 6481-6485, 2008).

The derived nucleotide sequences may differ from the reference sequence by substitution, deletion and/or insertion of one or more nucleotides, and this at positions such that these modifications do not significantly affect the activity of the protein coded by the nucleic acid. By <<a sequence at least 95% (for example) identical to a reference sequence>> is meant a sequence identical to the reference sequence except that this sequence may include up to five mutations (substitutions, deletions and/or insertions) for each portion of a hundred nucleotides of the reference sequence. Thus, for a reference sequence with 100 nucleotides, a fragment of 95 nucleotides and a sequence of 100 nucleotides including 5 substitutions relatively to the reference sequence are two examples of sequences 95% identical with the reference sequence. The identity percentage is generally determined by using a sequence analysis software package (for example the Sequence Analysis Software Package of the Genetics Computer Group, University of Winconsin Biotechnology Center, 1710 University Avenue, Madison, Wis. 53705). Preferably, the substitutions, deletions and/or insertions at the nucleotide sequence do not lead to a change of reading phase, nor to the introduction of a stop codon. The substitutions may either be silent or lead to mutations at the protein coded by the nucleic acid.

The protein binding a nucleic acid of interest may correspond to any protein capable of binding a nucleic acid in a non-covalent way. One skilled in the art may easily determine whether a protein is capable of binding a nucleic acid by the gel shift technique (see Examples 1.1.13. and 1.2.1.).

The protein binding a nucleic acid of interest may bind the nucleic acid of interest either in an aspecific way (i.e. it is capable of binding nucleic acids independently of their sequence), or in a sequence-specific way (i.e. it is only capable of binding to nucleic acids containing a particular sequence).

The mitochondrial targeting sequence is fused with the protein binding a nucleic acid of interest so that only a single protein is translated. In other words, the nucleic acid coding for the mitochondrial targeting sequence is fused with the acid coding for the protein binding a nucleic acid of interest so that there is a same and single open reading phase.

The fusion protein according to the invention includes the mitochondrial targeting sequence fused at the N-terminal end of the protein binding a nucleic acid of interest.

In a preferred embodiment according to the invention, the protein binding a nucleic acid of interest is the murine DHFR protein, which binds nucleic acids in an aspecific way. By <<DHFR>> is meant here the protein either coded by the sequence SEQ ID NO: 1, or by nucleotide sequence derived from SEQ ID NO: 1. Such derived nucleotide sequences may for example correspond to:
a fragment of at least 50, 100, 150, 200, 250, 300, 350, 400, 450, 500 or 550 nucleotides of the sequence SEQ ID NO:1;
a sequence at least 80%, 85%, 90%, 95%, 96%, 97%, 98% or 99% identical to SEQ ID NO:1;
an allelic variant of the sequence SEQ ID NO:1;
a homologous sequence to DHFR from a species other than mice;
   provided that the proteins coded by such derived nucleotide sequences retain their capability of binding nucleic acids. When the invention is applied with the DHFR protein, the nucleic acids of interest may have any sequence.

In another preferred embodiment according to the invention, the protein binding a nucleic acid of interest is the coat protein of the phage MS2, which binds nucleic acids in a sequence-specific way. By <<coat protein of the phage MS2>> or <<CP>> is meant here the protein either coded by the nucleotides 208 to 564 of the sequence SEQ ID NO: 18, or by nucleotide sequences derived from the nucleotides 208 to 564 of the sequence SEQ ID NO:18. Such derived nucleotide sequences may for example correspond to:
a fragment of at least 50, 75, 100, 125, 150, 175, 200, 250, 300, or 350 nucleotides of the nucleotides 208 to 564 of the sequence SEQ ID NO:18;
a sequence at least 80%, 85%, 90%, 95%, 96%, 97%, 98% or 99% identical to the nucleotides 208 to 564 of the sequence SEQ ID NO:18;
an allelic variant of the nucleotides 208 to 564 of the sequence SEQ ID NO:18;
a homologous sequence to CP from a species other than the phage MS2;
   provided that the proteins coded by such derived sequences retain their capability of binding nucleic acids in a sequence-specific way.

The coat protein of the phage MS2 recognizes the stem-loop region of the MS2 RNA. The term of <<stem-loop region of the MS2 RNA>> designates the nucleotides 154 to 172 and/or the nucleotides 193 to 211 of the sequence SEQ ID NO: 17. Consequently, when the invention is applied with the coat protein of the phage MS2, the nucleic acid of interest should contain or be fused with at least one stem-loop region of the MS2 RNA. The nucleic acid of interest may for example contain or be fused with at least one copy of the nucleotides 154 to 172 of the sequence SEQ ID NO:17, of the nucleotides 193 to 211 of the sequence SEQ ID NO:17, or of the nucleotides 124 to 239 of the sequence SEQ ID NO:17.

However, one skilled in the art may build a fusion protein according to the invention containing a protein binding a nucleic acid of interest in a sequence-specific way which is different from the coat protein of the phage MS2. For example, the protein binding a nucleic acid of interest may correspond to PUMILIO1 or to one of its fragments binding nucleic acids (Ozawa et al., Nature Methods, 4, 413-419, 2007). These may also be proteins such as transcription factors which specifically bind patterns known to one skilled in the art, patterns which may be easily found in the Interpro, Pfam or further SCOP data banks. For instance, let us mention the bZip proteins of Antirrhinum majus which preferentially bind CACGTG or TGACGT/C patterns (Martinez-Garcia et al., The Plant J, 13, 489-505).

A particularly preferred embodiment deals with a fusion protein comprising or consisting of a mitochondrial targeting sequence fused with the coat protein of the phage MS2. The mitochondrial targeting sequence preferentially corresponds to the mitochondrial targeting sequence pSu9. Consequently, the object of the invention is a fusion protein pSu9-CP comprising or consisting of a fragment of at least 50, 75, 100, 125, 150 or 175 amino acids of SEQ ID NO:19, or comprising or consisting of a sequence at least 80%, 85%, 90%, 95%, 96%, 97%, 98% or 99% identical to the sequence SEQ ID NO:19.

Another particularly preferred embodiment deals with a fusion protein comprising or consisting of a mitochondrial targeting sequence fused with the DHFR protein. The mitochondrial targeting sequence preferentially corresponds to the mitochondrial targeting sequence pSu9. Consequently, the object of the invention is a fusion protein pSu9-DHFR comprising or consisting of a sequence at least 80%, 85%, 90%, 95%, 96%, 97%, 98% or 99% identical to the sequence SEQ ID NO: 3 or comprising or consisting of a fragment of at least 50, 75, 100, 125, 150, 200 or 250 amino acids of SEQ ID NO: 3.

The fusion proteins according to the invention which comprise or consist of a sequence at least 80%, 85%, 90%, 95%, 96%, 97%, 98% or 99% identical to the sequence SEQ ID NO: 3 or 19 may contain mutations such as deletions, insertions and/or substitutions of amino acids. In a preferred embodiment, these proteins differ from proteins of sequence SEQ ID NO: 3 or 19 by conservative substitutions.

The invention also relates to a nucleic acid coding for a fusion protein according to the invention, as well as to recombinant vectors comprising such a nucleic acid.

In recombinant vectors according to the invention, the nucleic acid coding for the fusion protein according to the invention is preferentially placed under the control of expression signals (for example promoter, “enhancer”, terminator, translation signals, for example including the 5′ and 3′ UTR regions), so as to form an expression cassette.

A preferred embodiment according to the invention deals with recombinant vectors for gene therapy. By <<recombinant vector for gene therapy>> is meant here any vector suitable for gene therapy. Such vectors are generally in the form of a recombinant virus and therefore correspond to viral vectors. The viral vector may be selected from an adenovirus, a retrovirus, in particular a lentivirus, and adeno-associated virus (AAV), a herpes virus, a cytomegalovirus (CMV), a virus of vaccine, etc., Advantageously, the recombinant virus is a defective virus. The term of <<defective virus>> designates a virus incapable of replicating in a target cell. Generally, the genome of defective viruses is devoid of at least the sequences required for replication of said virus in the infected cell. These regions may either be suppressed or made non-functional or further substituted with other sequences and in particular with the nucleic acid which codes for the peptide of interest. Nevertheless, preferably, the defective virus retains the sequences of its genome which are required for the encapsulation of the viral particles.

In a preferred embodiment of recombinant vectors for gene therapy, the vector contains a nucleic acid coding for a fusion protein between a mitochondrial targeting sequence (for example pSu9) and a protein which binds a nucleic acid in a sequence-specific way (for example the coat protein of the phage MS2).

2. The Use of Fusion Proteins According to the Invention and of Kits for Importing Nucleic Acids of Interest into Mitochondria

The present invention relates to the use in vivo or in vitro, of a fusion protein according to the invention (i.e. a fusion protein between a mitochondrial targeting sequence and a protein binding a nucleic acid of interest), or of a nucleic acid coding for said fusion protein, for importing said nucleic acid of interest into a mitochondrion.

As apparent, considering the examples, said fusion protein is not bound covalently to the nucleic acid of interest. In other words, within the scope of the present invention, the fusion protein is naturally capable of binding the nucleic acid of interest without it being necessary to apply a chemical reaction so as to generate covalent bonds between the fusion protein and the nucleic acid of interest.

The nucleic acid of interest according to the invention may be any type of nucleic acid. This may be a single strand or double strand molecule of DNA or RNA nature and with a mitochondrial, plastidial or cytoplasmic origin. For instance, the nucleic acid of interest according to the invention may be an antisense RNA, or a messenger RNA (mRNA) or further a complete or partial transfer RNA (tRNA). The shuttle system according to the invention has allowed the transport of nucleic acids of different sizes, including nucleic acids of large size such as a transcript of 775 nucleotides (see Example 1.2.5.). Consequently, in a particular embodiment, the nucleic acid of interest has a size larger than 24, 50, 100 or 500 bases or base pairs. In a preferred embodiment, the nucleic acid of interest is a complete messenger RNA or a complete transfer RNA, in particular a messenger RNA including the 5′ and 3′ UTR regions.

By “in vitro” is meant here any method which is not carried out on a pluricellular organism such as an animal and/or human organism. On the other hand, the in vitro methods include methods carried out on cells, tissues or organs isolated beforehand from an animal and/or human pluricellular organ. The in vitro methods also include methods carried out on plant cells or tissues.

In preferred embodiments of the invention, said fusion protein comprises or consists of the mitochondrial targeting sequence pSu9 fused with the protein DHFR, or the mitochondrial targeting sequence pSu9 fused with the coat protein of the phage MS2.

The mitochondria may either be isolated, or be present within a cell. One skilled in the art may easily obtain isolated mitochondria for example by using the procedures described in Examples 1.1.4. and 1.1.6. The mitochondria may stem from any eukaryotic organism such as yeasts, fungi, plants or animals (including humans).

When the mitochondria are isolated, the fusion protein may bind the nucleic acid in an aspecific way or in a sequence-specific way. In the case of import into a mitochondrion of a cell, a fusion protein binding the nucleic acid in a sequence-specific way is preferentially used.

One skilled in the art may easily check whether the nucleic acid of interest has been imported into the mitochondrion for example by using the procedure described in Example 1.1.7.

The invention also relates to a kit for importing a nucleic acid of interest into a mitochondrion comprising:
i. a fusion protein according to the invention, or a nucleic acid coding for said fusion protein; and, optionally,
ii. at least one reagent for importing the nucleic acid of interest into the mitochondrion; and/or
iii. instructions for importing a nucleic acid of interest into a mitochondrion.

The reagents of the kit may correspond to any of the reagents described in the examples of the present application. For example they may correspond to at least one reagent selected from an import buffer (e.g. 600 mM mannitol, 2 mM potassium phosphate pH 7.5, 20 mM Hepes-KOH pH 7.2, 40 mM KCl, 2 mM DTT, 2 mM malate, 2 mM NADH), a wash buffer (e.g. 300 mM saccharose, 10 mM potassium phosphate pH 7.5, 1 mM EDTA, 0.1% (w/v) BSA, 5 mM glycine) and a STOP buffer (e.g. the wash buffer containing 5 mM EGTA and 5 mM EDTA). The composition of these reagents may vary depending on the organism from which stems the mitochondrion or on the cell containing the mitochondrion.

3. A Method for Importing a Nucleic Acid of Interest into an Isolated Mitochondrion

The present application also relates to a method for importing in vitro a nucleic acid of interest into an isolated mitochondrion, comprising the step of contacting a fusion protein according to the invention with a nucleic acid of interest and an isolated mitochondrion, thereby allowing non-covalent binding of the nucleic acid to the fusion protein.

The contacting may for example be achieved by mixing about 1 μg of the fusion protein with about 50 to 100 fmol of the nucleic acid of interest and about 200 μg of isolated mitochondria. To this mixture are added suitable reagents, such as an import buffer (for example containing 600 mM mannitol, 2 mM potassium phosphate pH 7.5, 2 mM Hepes-KOH pH 7.2, 20 mM, 40 mM KCl, 2 mM DTT, 2 mM malate and 2 mM NADH), ADP and ATP.

After the contacting, they are left in contact so that the nucleic acid of interest is imported into the isolated mitochondrion. To do this, they are incubated at a suitable temperature and for a suitable duration. For example, the incubation may be carried out between 4° C. and 30° C., between 20° C. and 30° C., or preferentially around 25° C. The incubation may for example have a duration of 5 mins to 16 h (overnight), 15 mins to 2 h, or about 30 mins.

The method may include an additional step consisting of adding a mixture containing RNAse or DNAse in order to degrade the nucleic acids of interest found outside the mitochondria.

The method may contain another additional step which consists of stopping the reaction, centrifuging the mixture, removing the supernatant and washing the pellet of mitochondria.

The nucleic acids of the pellet of mitochondria may then be extracted for analysis.

When the import method according to the invention is applied with a nucleic acid coding for the fusion protein according to the invention, the import method further contains the following steps, before step (a):
producing a fusion protein according to the invention; and optionally
purifying said fusion protein.

The fusion protein according to the invention may for example be produced with recombinant techniques, for example by expressing the nucleic acid coding for a fusion protein according to the invention in a host cell suitable for producing recombinant proteins. Such host cells notably include bacteria (E. coli), yeasts, fungi, baculovirus host cells, as well as insect cells, plant, animal and/or human cells.

4. A Method for Importing a Nucleic Acid of Interest into the Mitochondrion of a Cell

One aspect of the invention relates to the import of nucleic acids of interest in a sequence-specific way. In this case, the cell is transformed by two nucleic acids, one being the nucleic acid of interest and the other coding for a fusion protein according to the invention binding a nucleic acid in a sequence-specific way. Once they are integrated into the nuclear genome of the cell, both nucleic acids are transcribed into RNA. The RNA coding for the fusion protein according to the invention is translated into a protein. Once it is expressed in the cytoplasm of the cell, the fusion protein then allows import of the nucleic acid of interest, itself also present in the cytoplasm, into the mitochondrion.

Thus, the invention relates to a combination of nucleic acids comprising:
a) a nucleic acid coding for a fusion protein between a mitochondrial targeting sequence and a protein binding a nucleic acid in a sequence-specific way; and
b) a nucleic acid of interest fused with the nucleic acid bound by said protein binding a nucleic acid in a sequence-specific way, or a nucleic acid, whose transcription produces such a nucleic acid.

In a preferred embodiment, the combination of nucleic acids comprises:
a) a nucleic acid coding for the mitochondrial targeting sequence (for example pSu9) fused with the coat protein of the phage MS2; and
b) a nucleic acid of interest fused with at least one stem-loop region of the MS2 RNA.

In a first embodiment, a single and same recombinant vector comprises the nucleic acids (a) and (b). In other words, the combination corresponds to a recombinant vector comprising the nucleic acids (a) and (b). Such a vector is part of the recombinant vectors according to the invention.

Alternatively, both nucleic acids are positioned on two different vectors. In this case, the combination corresponds to a combination of recombinant vectors, one comprising the nucleic acid (a), the other one comprising the nucleic acid (b).

The invention also relates to a method for importing in vitro or in vivo a nucleic acid of interest into a mitochondrion of a cell, comprising the steps:
a) obtaining or preparing a combination of nucleic acids as defined above; and
b) introducing said combination of nucleic acids into a cell.

The nucleic acid coding for the fusion protein according to the invention is preferentially placed under the control of expression signals (e.g. a promoter, <<enhancer>>, terminator, translation signals, 5′ or 3′ UTR), so as to form an expression cassette. As to the nucleic acid of interest fused with the nucleic acid recognized by said protein binding a nucleic acid in a sequence-specific way, it is preferentially placed under the control of signals allowing its transcription (e.g. promoter, <<enhancer>>, terminator).

The cell may correspond to any eukaryotic cell containing mitochondria, for example cells of yeast, of fungi, of protozoans, of plants or animals (including human cells).

The vectors may be introduced into the cell through any suitable method, such as electroporation, biolistic transformation, transformation via an agrobacterium or any bacterial agent adapted to the host organism, micro-injection, chemical methods, etc., The method will generally be selected depending on the type of cell used.

5. Fields of Application of the Invention

The fusion proteins, nucleic acids, vectors, uses and methods described above are notably useful in the field of gene therapy, in agronomics, for producing proteins in mitochondria, for analyzing fundamental processes for mitochondrial biogenesis, and for manipulating the expression of the mitochondrial genome.

The fusion proteins, nucleic acids, vectors, uses and methods described above may for example be used for analyzing fundamental processes for mitochondrial biogenesis. The facility for introducing nucleic acids into mitochondria at will, regardless of their sequence and/or their size, gives the possibility of contemplating many studies aiming at comparing wild nucleic acids with mutated forms, at studying their stability and/or their functionality in the organelle.

The fusion proteins, nucleic acids, vectors, uses and methods described above also allow manipulation of the expression of the mitochondrial genome. Thus, the shuttle system according to the invention gives the possibility of easily and efficiently introducing antisense RNAs, oligonucleotides, sense or antisense DNAs or RNAs, ribozymes, mating regions involved in the replication transcription or translation in order to directly and rapidly test the effect of the expression on the mitochondrial genome, for example in plants.

On the other hand, the fusion proteins, nucleic acids, vectors, uses and methods described above allow the production of allogenic proteins. The present invention for the first time allows introduction of complete mRNAs with their 5′ and 3′ UTR regions into a mitochondrion. The production of the corresponding protein may then be contemplated. In the absence of sequences indispensable for eukaryotic translation, these mRNAs will exclusively be translated into protein in the mitochondrion through which they are addressed. If the genetic code diverges, this is the case for animal mitochondria for example, the mRNA may only be produced in the mitochondrion because of the codons used.

The present invention also relates to the use of fusion proteins according to the invention in the field of gene therapy. The nucleic acid of interest is imported into a mitochondrion of a human or animal cell by a fusion protein according to the invention.

Thus, an aspect of the invention deals with:
a recombinant vector according to the invention, or a combination of vectors or nucleic acids according to the invention for use as a drug and/or for use in gene therapy;
the use of a recombinant vector according to the invention, or of a combination of vectors or nucleic acids according to the invention, for preparing a drug, preferentially intended for gene therapy;
a pharmaceutical composition comprising a fusion protein according to the invention, a recombinant vector according to the invention, or a combination of vectors or nucleic acids according to the invention.

Recombinant vectors may correspond to any recombinant vector containing a fusion protein according to the invention. They preferentially correspond to vectors for gene therapy. The vectors and combinations of vectors or of nucleic acids preferentially correspond to those described in the paragraph 4 above, entitled <<A method for targeted and specific import of a nucleic acid of interest>>.

The vector(s) may be introduced in vivo by any technique known to one skilled in the art. In particular, it is possible to introduce the DNA vector in vivo in a naked form, i.e. without the assistance of any carrier or system which would facilitate transfection of the vector into the cells. A gene gun may also be used, for example by depositing DNA at the surface of <<gold>> particles and projecting the latter so that the DNA penetrates through the skin of a patient. Injections by means of a liquid gel are also possible for transfecting both the skin, muscle, fat tissue and breast tissue. Microinjection techniques, electroporation, precipitation with calcium phosphate, formulations by means of nanocapsules or liposomes are other available techniques. Biodegradable nanoparticles in polyalkyl cyanoacrylate are particularly advantageous. In the case of liposomes, the use of cationic lipids promotes encapsulation of the nucleic acids which are negatively charged and facilitates fusion with the negatively charged cell membranes. A targeted administration of genes is for example described in application WO 95/28 494. In plants, the DNA may be conventionally transferred via the use of Agrobacterium tumefaciens.

The vectors and combinations according to the invention may be used for treating any human disease related to mitochondrial deficiency. The vectors and combinations allow complementation of the deficient gene by introducing via transgenesis the non-mutated gene into the nuclear genome, while presently direct genetic transgenesis of human mitochondria is impossible. The product of these genes is then addressed to the mitochondrion. The present invention gives the possibility of considerably widening a range of nucleic acids which may be transported into the mitochondrion. Further, it gives the possibility of very rapidly testing in vitro in isolated mitochondria, the most suitable nucleic acids for complementing a deficient mitochondrial function, and/or for inhibiting replication of <<diseased>> nucleic acid molecules in order to switch the critical threshold towards <<healthy>> nucleic acid molecules.

An example of a disease which may be treated with recombinant vectors and combinations according to the invention is the MERRF (<<Myoclonic Epilepsy and Red Ragged Fibers>>) syndrome. The invention therefore deals with a recombinant vector according to the invention or a combination of vectors or nucleic acids according to the invention for treating and/or preventing the MERRF syndrome. In this case, the nucleic acid of interest which is present in the vector or the combination according to the invention is the human mitochondrial tRNALys.

The present invention finally relates to the use of fusion proteins according to the invention in agronomics. The nucleic acid of interest is imported into a mitochondrion of a plant cell by means of a fusion protein according to the invention which preferably binds said nucleic acid of interest in a sequence-specific way.

Thus, an aspect of the invention deals with the use in vitro of the vector according to the invention, or of a combination of vectors or nucleic acids according to the invention, for importing said nucleic acids of interest into a mitochondrion of a plant cell. The plant cell may for example correspond to a protoplast. The recombinant vectors may correspond to any recombinant vector containing a fusion protein according to the invention. The vectors and combinations of vectors or nucleic acids preferentially correspond to those described in paragraph 4 above, entitled <<Targeted and specific method for importing a nucleic acid of interest>>.

The invention more particularly relates to a method for obtaining a recombinant plant characterized in that it includes the following steps:
a) obtaining or preparing a combination of vectors or nucleic acids according to the invention;
b) introducing said combination of vectors or nucleic acids in a plant cell, for example by transforming a protoplast;
c) regenerating an entire plant from the recombinant plant cell obtained in step (b); and
d) selecting the plants having integrated into their genome, said vectors or nucleic acids.

Moreover, it is possible to inject isolated mitochondria into plant protopoplasts (Verhoeven et al., Plant Cell Reports, 14: 781-785, 1995). Consequently, the invention also relates to a method for obtaining a recombinant plant characterized in that it includes the following steps:
a) putting a fusion protein according to the invention in contact with the nucleic acid of interest and an isolated mitochondrion; and
b) leaving them in contact so that the nucleic acid of interest is imported into the isolated mitochondrion;
c) introducing said isolated mitochondrion into a plant cell, for example a protoplast;
d) regenerating an entire plant from the recombinant plant cell obtained in step (c); and
e) selecting the plants having integrated into their genome said nucleic acid of interest.

The above method may also be applied both with fusion proteins according to the invention binding the nucleic acid of interest in a sequence-specific way and with fusion proteins according to the invention binding the nucleic acid of interest in an aspecific way.

The plants obtained in step (d) or (e) of the above methods may then be cross-bred with each other and homozygous plants for the nucleic acid of interest may be selected. Alternatively, the plants obtained in step (d) or (e) may be cross-bred with a plant of the same species, and the plants stemming from the cross-breeding and having retained the nucleic acid of interest may be selected.

The recombinant plants obtained with said methods are also part of the invention, as well as the seeds and fruit of such plants.

These methods are useful for improving the characteristics of plants of agronomic interest, for example their resistance to biotic and/or abiotic stresses and for controlling cytoplasmic male sterility. In the latter case, the methods according to the invention are used for introducing an mRNA producing a protein capable of generating cytoplasmic male sterility. Thus, a preferred embodiment of the invention relates to the use in vitro of a vector according to the invention, or of a combination of vectors or nucleic acids according to the invention for generating cytoplasmic male sterility in a plant.

All the articles, journals, patent applications, patents and handbooks mentioned herein are incorporated by reference to the text of the present application.

Although having distinct meanings, the terms of <<comprising >>, <<containing>>, <<including>> and <<consisting in>> have been used in an interchangeable way in the description of the invention and may be replaced with each other.

The following examples and figures illustrate the invention without limiting the scope thereof.

### DESCRIPTION OF THE FIGURES

FIG. 1: A. Interaction of the protein pSu9-DHFR with the radioactive transcript corresponding to cytosolic tRNAAla of Arabidopsis thaliana. The analysis was carried out with the gel shift technique. The figure shows an autoradiograph of the gel in the native condition. The first column (−) shows the radio-labelled tRNAAla used as a probe. The other columns (DHFR) show that delayed migration of the probe is observed in the presence of an increasing amount of DHFR. B. Effect of the addition of pSu9-DHFR on the import in vitro of the transcript of radioactively labelled tRNAAla in isolated potato mitochondria. The figure illustrates the autoradiograph of a 15% denaturing polyacrylamide gel. <<In>> represents the initial amount of tRNAAla transcript present in the import medium. <<+>> represents the amount of tRNAAla transcript in the presence of 1 μg of pSu9-DHFR. <<−>> represents the amount of tRNAAla transcript in the absence of pSu9-DHFR. Two exposures of the same gel (4 h and overnight) are shown.

FIG. 2: A. Effect of the addition of pSu9-DHFR and of DHFR on the in vitro import of the radioactively labeled transcript of tRNAAla in isolated potato mitochondria. The figure shows the autoradiograph of a 15% denaturing polyacrylamide gel. <<In>> represents the initial amount of tRNAAla transcript present in the import medium. <<−>> represents the amount of tRNAAla transcript in the absence of DHFR or of pSu9-DHFR. <<2>> represents the amount of tRNAAla transcript in the presence of 2 μg of DHFR or of pSu9-DHFR. <<4>> represents the amount of tRNAAla transcript in the presence of 4 μg of DHFR or of pSu9-DHFR. B. Effect of the addition of pSu9-DHFR on the in vitro import of the radioactively labeled tRNAAla transcript in isolated potato mitochondria in the presence of methotrexate. The figure shows the autoradiograph of a 15% denaturing polyacrylamide gel. <<In >> represents the initial amount of tRNAAla transcript present in the import medium. <<−>> represents the amount of tRNAAla transcript in the absence of pSu9-DHFR. <<+>> represents the amount of tRNAAla transcript in the presence of 1 μg of pSu9-DHFR. “Mtrx” represents the amount of tRNAAla transcript: in the presence of 1 μg of pSu9-DHFR and of methotrexate (50 μM).

FIG. 3: Effect of the addition of pSU9-DHFR on the in vitro import of the transcript corresponding to the edited or non-edited larch tRNAHis precursor in isolated potato mitochondria. A. Diagram of the larch tRNAHis precursor. P1 and P2 indicate the schematic localization of the hybridation sites of the primers used for cRT-PCR. B. Import of the radioactively marked transcript. The figure represents the autoradiograph of a 15% denaturing polyacrylamide gel. <<In >> represents the initial amount of precursor tRNAHis transcript present in the import medium. <<−>> represents the amount of precursor tRNAHis transcript in the absence of pSU9-DHFR. <<+>> represents the amount of precursor tRNAHis transcript in the presence of 1 μg of pSu9-GFP. <<pretrnH-ed >> designates the edited tRNAHis precursor. <<pretrnH-uned >> designates the non-edited tRNAHis precursor. <<tr >> designates the mature tRNAHis transcript. C. Sequence of 15 clones of tRNAHis obtained after import in vitro. Only the 5′ and 3′ ends of the sequences are illustrated. The CCA sequence added after transcription, observed for 5 of these clones, is illustrated in underlined text in italics. The sequences of the non-completely matured precursor are illustrated in bold characters.

FIG. 4: Effect of the addition of pSu9-DHFR on the in vitro import of an oligigonucleotide corresponding to the radioactively labeled tRNAAla in isolated potato mitochondria. The figures show the autoradiograph of a 15% denaturing polyacrylamide gel. <<T >> represents the initial amount of tRNAAla transcript present in the import medium. <<+>> represents the amount of tRNAAla transcript in the presence of 1 μg of pSu9-DHFR. <<−>> represents the amount of tRNAAla transcript in the absence of pSu9-DHFR. <<Mi >> designates the RNase treatment carried out on integral mitochondria. <<Mtp >> designates the RNase treatment carried out on mitoplasts. A. Isolated potato mitochondria. B. Isolated yeast mitochondria.

FIG. 5: Import of RNA into the mitochondria in the presence of pSu9-MS2. A. Effect of the addition of MS2 or pSu9-MS2 on the import of the radioactive transcript corresponding to tRNAHis fused with the stem-loop RNA region of the phage MS2 in potato mitochondria. Autoradiograph of a 8% denaturing polyacrylamide gel. −: absence of MS2 or pSu9 protein, +: in the presence of 1 μg of MS2 or pSu9-MS2. B. Inducible and stable expression of yeast cells of S. cervisiae transformed with the vector pESC TRP either including or not the gene coding for MS2 or pSu9-MS2 and the gene coding for the anticox-2SL construct. Western blot analysis of the expression of the protein in a total extract of yeast proteins and analysis by RT-PCR of the expression of the RNA in a total yeast RNA extract. −: yeasts transformed with the vector pESC TRP alone; M: transformation with the vector including the gene coding for the anticox-2SL RNA and for the MS2 protein; pM: transformation with the vector including the gene coding for the anticox-2SL RNA and for the protein preMS2. The proteins are revealed by means of an anti-Myc. Antibody. p: pSu9-MS2 form, m: MS2 form. The PCR product obtained after amplification with RT-PCR by means of a specific pair of oligonucleotides is viewed with an arrow. *: aspecific amplication product. C. Western blot and Northern blot analysis of the quality of the preparations of yeast mitochondria. An antibody directed against by the protein Kar2 of the endoplasmic reticulum shows the absence of contamination of the mitochondrial preparations. An oligonucleotide probe directed against the cytosolic yeast trK2 tRNALys(UUU) shows the absence of RNA contaminants in the mitochondrial preparations. Total: extract of total yeast proteins or of total yeast RNA; Mito: extract of yeast mitochondrial proteins or yeast mitochondrial RNA. −: yeasts transformed with the vector pESC TRP alone; M: transformation with the vector including the gene coding for the anticox-2SL RNA and for the MS2 protein; pM: transformation with the vector including the gene coding for the anticox-2SL RNA and for the preMS2 protein. D. Analysis of the import of the MS2 protein and of the anticox-2SL RNA into yeasts either transformed or not. Western blot analysis for MS2 by means of an antibody directed against the Myc tag fused with the protein by cloning in the vector pESC TRP and analysis by RT-PCR for the anticox-2SL RNA with a specific pair of primers. −: transformed yeasts with the vector pESC TRP alone; M: transformation with the vector including the gene coding for the anticox-2SL RNA and for the MS2 protein; pM: transformation with the vector including the gene coding for the anticox-2SL RNA and for the pre MS2 protein. The PCR product obtained after application with RT-PCR by means of a specific pair of oligonucleotides is viewed with an arrow.

### DESCRIPTION OF THE SEQUENCES OF THE LIST OF SEQUENCES

SEQ ID NO: 1 illustrates the sequence coding for mouse DHFR.

SEQ ID NO: 2 illustrates the mitochondrial targeting sequence pSu9 of the atp9 gene of Neurospora crassa.

SEQ ID NO: 3 represents the protein sequence of the fusion protein pSu9-DHFR SEQ ID NO: 4 represents the sequence coding for cytosolic tRNAAla of Arabidopsis thaliana.

SEQ ID NO: 5 represents the sequence coding for GFP.

SEQ ID NO: 6 represents the sequence coding for the precursor form of larch mitochondrial tRNA(His). The nucleotides in positions 81, 87 and 117 are edition sites.

SEQ ID NOS. 7 and 8 represent primers for analysis by the cRT-PCR technique.

SEQ ID NO: 9 represents the sequence coding for potato mitochondrial atp9. The nucleotides in positions 353, 383, 414, 415, 423, 425, 515, 524, 545 and 556 correspond to edition sites. The non-edited version of the gene coding for mitochondrial atp9 contains a cytidine at the positions noted as <<y>>. At the corresponding mRNA obtained after transcription, these positions are edited into uridine with mitochondrial matrix enzymes (generating thymides at the corresponding DNA sequences).

SEQ ID NO: 10 represents the sequence coding for the potato mitochondrial atp9 alone. This sequence contains the same edition sites as sequence SEQ ID NO: 9.

SEQ ID NOS. 11 to 14 represent oligonucleotides for mutagenesis by PCR.

SEQ ID NOS. 15 and 16 represent primers used for analysis by RT-PCR.

SEQ ID NO: 17 represents the sequence coding for the precursor form of larch mitochondrial tRNAHis fused on side 3′ with the sequence coding for the stem-loop portion of the MS2 RNA.

SEQ ID NO: 18 represents the nucleotide sequence corresponding to the mitochondrial targeting sequence pSu9 fused with the CP protein.

SEQ ID NO: 19 represents the polypeptide sequence of the mitochondrial targeting sequence pSu9 fused with the CP protein.

SEQ ID NOS. 20 to 27 represent oligonucleotides for mutagenesis by PCR.

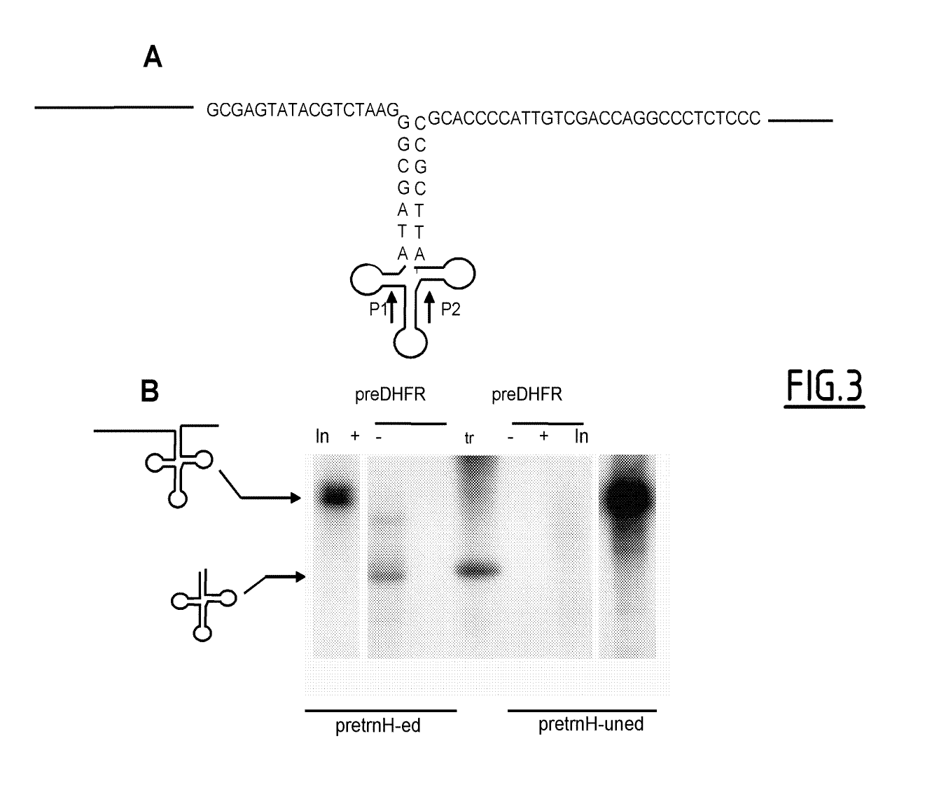

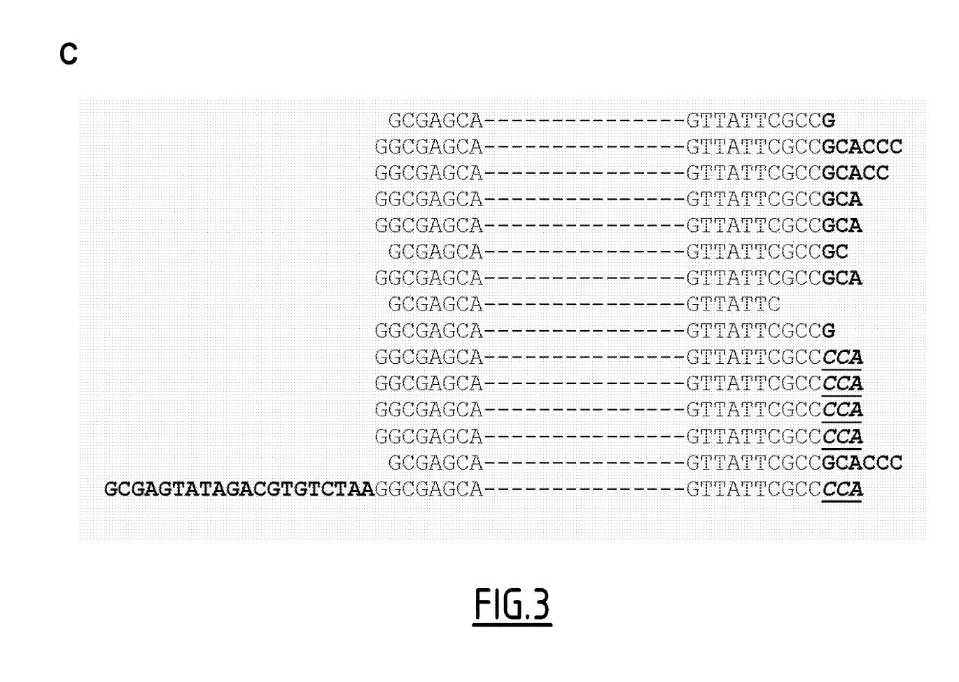

SEQ ID NOS. 28 to 34 represent the sequences of at least ten amino acids shown in FIG. 3C.

SEQ ID NO: 35 represents the sequence called anticox-2SL, which comprises the anti-sense region of the promoting region of the yeast mitochondrial gene COX 1, fused on the 3′ side with the sequence of the stem-loop portion of the MS2 RNA.

SEQ ID NO: 36 represents the oligonucleotide primer No. 1 of anticox-2SL.

SEQ ID NO: 37 represents the oligonucleotide primer No. 2 of anticox-2SL.

SEQ ID NO: 38 represents an oligonucleotide sequence complementary to the yeast trK3 mitochondrial tRNALys.

SEQ ID NO: 39 represents an oligonucleotide sequence complementary to the yeast trK2 cytosolic tRNALys.

### Example 1

### Allogenic RNA Targeting In Vitro in Isolated Mitochondria of Plants Via the pSu9-DHFR Protein Shuttle

1.1. Material and Methods

1.1.1. Over Expression and Purification of the Recombinant Protein pSu9-DHFR in Escherichia coli

The vector pQE40 (Quiagen) containing a chimeric gene coding for mouse DHFR (SEQ ID NO: 1) fused with the mitochondrial targeting sequence pSu9 of the atp9 gene of Neurospora crassa (SEQ ID NO: 2; Pfanner et al., Eur J Biochem, 169, 289-293, 1987) was used for overexpression of the fusion protein pSu9-DHFR (SEQ ID NO: 3). This vector pSu9-DHFRpQE40 is used for transforming E. coli strain M15 bacteria.

pSu9-DHFR may easily be purified in a native condition by means of a histidine tag present at the C terminal end of the protein. The following procedure was used. 5 mL of LB medium added with ampicillin (100 μg/mL) are sown with the recombinant strain M15 of E. coli containing the plasmid pSu9-DHFRpQE40 and are incubated overnight at 37° C. with stirring. This preculture is used for sowing 45 mL of LB (yeast extract 5 g/L, bactotryptone 10 g/L, NaCl 10 g/L, pH 7.0) added with ampicillin (100 μg/mL) and with IPTG (isopropyl-b-D-galactothiopyranoside) (1 mM). The IPTG induces expression of the protein. The overexpression is achieved for 4 hours at 37° C. with stirring. The bacteria are recovered by centrifugation for 5 minutes at 11,000 g at room temperature.

All the steps are performed at 4° C. After obtaining the induced bacteria, the latter are subject to 10 sonications each with a duration of 8 seconds in 1.8 mL of lysis buffer (50 mM Tris-malate pH 8.2, 0.6M NaCl, 5 mM MgCl₂, 1% (v/v) glycerol, 1% (v/v) Triton X-100, 70 mM imidazole, 20 μL of 100 mM PMSF (paramethyl sulfonyl fluoride) added extemporaneously). The cell lysate is centrifuged for 30 minutes at 4,000 g. 200 μL of Ni-NTA Superflow Resin (Qiagen) are washed 3 times in 500 μL of wash buffer (50 mM Tris-malate pH8.2, 0.6M NaCl, 5 mM MgCl₂, 1% (v/v) glycerol, 70 mM imidazole. The resin is recovered between each wash by centrifugation for 5 minutes at 9,000 g. The supernatant of the cell lyzate is added to the resin. The whole is incubated for 1 hour with gentle stirring. After 5 minutes of centrifugation at 9,000 g the supernatant is removed. The resin is washed 3 times in 300 μL of wash buffer as previously. The proteins bound on the resin are then eluted in the presence of 100 μL of elution buffer (25 mM Hepes pH 7.5, 0.6M NaCl, 5 mM MgCl₂, 250 mM imidazole) for 15 minutes. The eluate is recovered by centrifugation for 5 minutes at 9,000 g. This eluation step is repeated twice and the eluates are frozen in liquid nitrogen and then kept at −80° C.

1.1.2. DNA Transcription In Vitro

The Riboprobe® kit (Promega) allows production of transcripts in vitro from the pGEM®-T Easy vector containing the genes of interest and linearized by a restriction enzyme. Depending on the orientation of the insert in the vector, the RNA polymerases T7 or Sp6 are used. The reaction takes place for 3 hours at 37° C. in 20 μL of the following reaction medium: 5 to 10 μg of digested plasmid DNA, 4 μL of transcription buffer 5× (400 mM Hepes-KOH pH 7.5, 120 mM MgCl₂, 200 mM DTT, 10 mM spermidine), a mixture of NTP**, 1 μL of an enzyme mixture (15-20 U of RNA polymerase T7 or Sp6, 0.3 U of pyrophosphatase) and 40 U of an inhibitor of RNases (RNaseOUT™-Invitrogen).

After the reaction for synthesizing the transcripts, 2 units of DNase RQ1 (Promega) as well as 5 μL of DNase buffer 10× (200 mM Tris-HCl pH 8.0, 100 mM MgCl₂) are added and the volume is completed to 50 μL. An incubation at 37° C. for 15 minutes is required for digesting the DNA. In order to remove the nucleotides not incorporated during the transcription in vitro, and the DNA degradation products, the reaction medium is deposited on a 1 mL Sephadex G-50 column dried before hand by centrifugation at 200 g. The elution is accomplished by fresh centrifugation under the same conditions. The RNAs are then precipitated at −20° C. for one hour from 2.5 volumes of ethanol in the presence of 0.1 volume of 1M sodium acetate pH 4.8. After centrifugation for 20 minutes at 16,000 g, the pellet is dried in open air and taken up in 10 μL of water.

Three types of RNA may be produced depending on the NTP mixture used. Non-labeled RNA transcripts are made in a reaction medium containing 2.5 mM of each NTP. Radioactively marked RNAs are produced in a reaction medium containing 2.5 mM of GTP, CTP and ATP, 0.5 mM of UTP and 4 μL of [α³²P]UTP (10 μCi/μL, specific activity: 3 μCi/μmol). RNAs marked with a fluorophore are produced in a reaction medium containing 0.5 mM of GTP, CTP and ATP, 0.375 mM of UTP and 100 μM of UTP Chromatide® Alexa Fluor® 488 (Molecular Probe).

1.1.3. Mutagenesis by PCR—Construction of Chimeric Genes

Chimeric genes are constructed with the extension technique of overlapping fragments by PCR. This method requires two steps. During the first step, two PCRs are carried out in parallel from two different templates: template No. 1 in the presence of the first pair of primers (primers a and b) and template No. 2 in the presence of the second pair of primers (primers c and d). One tenth of both PCR products obtained are then put into the presence of the primers a and d for carrying out a last PCR step. Finally, the PCR product corresponds to the fusion of one portion of the two initial templates.

1.1.4. Purification of Potato Mitochondria

The procedure used is the one described by Pujol et al. (Proc Natl Acad Sci, 105, 6481-6485, 2008). The yield is of about 15 mg of equivalent of mitochondrial proteins per kg of potato tubers (Bintje variety). When they are used for import experiments, the mitochondria are put back into a minimum PDT wash buffer volume.

1.1.5. Assaying the Mitochondria in Protein Equivalents by the Bradford Method

This method is based on the modification of the absorption wavelength of Coomassie Blue G-250 in an acid medium after its binding on the proteins (595 nm). In practice, 800 μL of Bradford reagent (BioRad) and 200 μL of protein extract to be quantitated are mixed and absorbance is measured at 595 nm. This OD measurement allows determination of the amount of proteins present by referring to a BSA standard range established with known amounts. The amount of mitochondria used during the different experiments was evaluated with this technique. In the present application <<mg of mitochondria >> refers to mg of mitochondrial protein equivalent.

1.1.6. Obtaining Mitoplasts

Mitoplasts are mitochondria in which there was a breakage of the external membrane. In order to prepare mitoplasts, a method based on the principle of hypotonic swelling is used. The change in osmolarity causes breakage of the external membrane, but not that of the internal membrane. The procedure followed for obtaining potato mitoplasts is the one described by Delage et al. (Mol Cell Biol, 23, 4000-4012, 2003).

1.1.7. In Vitro Import of Radiolabelled Proteins into Isolated Potato Mitochondria

In Vitro Transcription and Translation

The precursor of the protein for which import into the mitochondria is desirably studied, is synthesized by transcription and translation coupled in a lyzate of rabbit reticulocytes in the presence of methionine marked with [³⁵S]. We use the system <<TNT® Coupled with Reticulocytes Lyzate System >> (Promega). The 50 μL reaction medium contains 25 μL of TNT® Rabbit Reticulocytes Lyzate (Promega), 2 μL of transcription buffer TNT® (Promega), 1 μL of RNA polymerase TNT® (Promega) T7 or Sp6 depending on the orientation of the gene in the plasmid. 1 μL of a mixture of amino acids without 1 mM methionine, 2 μL of [³⁵S] methionine 10 μCi/μL (specific activity of 1,000 Ci/mmol), 1 to 2 μg of a plasmid containing the RNA polymerase promoter T7 or Sp6 and the complete cDNA of the protein of interest. After 1 hour 30 mins of incubation at 30° C., the whole is frozen to −80° C.

In Vitro Test for Importing Proteins into Mitochondria

The import in vitro of proteins into mitochondria is carried out according to the procedure described by Pujol et al. (Proc Natl Acad Sci, 105, 6481-6485, 2008). Fifty μg of potato mitochondria are taken up in a final volume of 50 μL containing 25 μL of import buffer (300 mM Mannitol, 20 mM Hepes-KOH pH 7.5, 80 mM KCl 1 mM K₂HPO₄, 1 mM, malate, 1 mM DTT, 1 mM NADH) in the presence of 40 μM ADP and 2 mM ATP. The radiolabelled proteins (5 μL) are added and the solution is incubated for 30 minutes at 25° C. with stirring. The reaction medium is then deposited on a 27% saccharose cushion (20 mM Tris-HCl pH 7.5, 27% w/v saccharose, 1 mM EDTA pH 8.0, 100 mM K₂HPO₄, 1 mg/mL BSA) and the mitochondria are recovered after centrifugation for 10 minutes at 9,000 g. The supernatant is removed and the pellet of mitochondria is analyzed on a denaturing polyacrylamide gel. The gel is then dried and then exposed against a Phosphorimager plate (Fuji) or subject to autoradiography.

In practice, an import experiment requires a certain number of controls. Four different tests are therefore generally carried out:
the first sample does not undergo any particular treatment;
the second sample undergoes treatment with proteinase K (100 μg/mL for 10 minutes at room temperature then inhibited with 1 mM PMSF). With this treatment, only the proteins incorporated into the mitochondria are then protected from the action of proteinase K;
in the third test, the mitochondria are pre-treated with valinomycin (2 μM) for 10 minutes at 4° C. before adding the labeled proteins. This treatment has the effect of dissipating the membrane potential thereby blocking transport of the precursor through the import channel of the proteins.
the fourth sample is treated with valinomycin and with proteinase K. Under these conditions, neither the precursor protein nor the mature protein are protected from degradation by proteinase K. This control shows that the band considered as the mature protein is not a degradation product which is resistant to proteinase K.

Protein Analysis on a Denaturing Polyacrylamide Gel

Separation of the proteins is carried out by electrophoresis on a 12% polyacrylamide gel under denaturing conditions in the presence of SDS. The gel includes a concentration gel (5% acrylamide/bisacrylamide 37.5/1 (w/v), 125 mM Tris-HCl pH 6.8, 0.1% (w/v) SDS) and a separation gel (12% acrylamide/bisacrylamide 35.5/1 (w/v), 380 mM Tris-HCl pH 8.8, 0.1% (w/v) SDS). Polymerization of the gel is obtained by adding 0.1% (w/v) ammonium persulfate and 0.01% (v/v) TEMED. A Laemmli buffer volume (100 mM Tris-HCl pH 6.8, 4% (w/v) SDS, 4% (v/v) β-mercaptoethanol, 15% glycerol, 0.05% (w/v) bromophenol blue) is added to the protein samples before deposition. Migration is accomplished in an SDS-PAGE buffer (25 mM Tris, 250 mM glycine, 0.1% (w/v) SDS) under a constant current of 30 mA. The proteins are revealed by incubation in a staining solution (Coomassie Blue at 0.25% (w/v), 10% (v/v) acetic acid, 40% (v/v) methanol) for 30 minutes and then by several successive passings through a discoloration solution (10% (v/v) acetic acid, 20% (v/v) ethanol). The gel is then dried for 1 hour in a gel dryer, before being exposed against a Phosphorolmager plate (Fuji) and/or subject to autoradiography in order to view the radioactive proteins.

Import of Nucleic Acids into the Mitochondria

An experiment for importing a radiolabelled transcript into potato mitochondria is conducted in a 100 μL reaction medium containing 50 μL of import buffer 2×PDT (600 mM mannitol, 2 mM potassium phosphate pH 7.5, 20 mM Hepes-KOH pH 7.2, 40 mM KCl, 2 mM DTT, 2 mM malate, 2 mM NADH), 40 μM ADP, 5 mM ATP, 5 mM MgCl₂, 200 μg of mitochondria (protein equivalent), 50 to 100 fmol of transcript labelled with [α³²P]UTP (50,000 to 100,000 cpm) and 1 μg of pSu9-DHFR. After incubation for 30 minutes at 25° C., 100 μL of an RNase mixture (100 μg/mL of RNase A, 750 U/mL of RNase T1 in a wash buffer: 300 mM saccharose, 10 mM potassium phosphate pH 7.5, 1 mM EDTA, 0.1% (w/v) BSA, 5 mM glycine) are added in order to degrade the molecules of transcripts which are outside the mitochondria. After incubation for 10 minutes at 4° C., 1 mL of STOP buffer (5 mM EGTA, 5 mM EDTA in a wash buffer) is added and the whole is centrifuged for 5 minutes at 9,000 g. The supernatant is removed and the pellet of mitochondria undergoes two other identical washing steps. The RNAs are then extracted and analysis of the radioactive transcripts is carried out on a denaturing polyacrylamide gel for small RNAs (75 nucleotides) or on an agarose formaldehyde gel for RNAs of larger size (500 to 1,000 nucleotides). The gel is then dried and then exposed against a plate of Phosphorimager (Fuji) or subject to autoradiography.

In every case, a control experiment carried out in the absence of pSu9-DHFR is conducted. It allows verification that the presence of pSu9-DHFR has a positive effect on the RNA transport. In order to validate the internalization of the RNAs in the template space, 100 μL of RNAse mixture may then be added after obtaining mitoplasts. In this case, the RNAs protected from the action of RNases have crossed the internal membrane and are present in the mitochondrial matrix.

During experiments in the presence of non-radiolabelled RNAs, internalization of the RNAs was evaluated by RT-PCR or by RT-PCR on circularized RNA (cRT-PCR).

During experiments in the presence of an RNA marked with a fluorophore, the mitochondria are viewed in confocal microscopy directly after the incubation step in the import medium.

1.1.8. Extraction of Mitochondrial RNAs

A pellet of potato mitochondria (200 μg of protein equivalent) taken up in 10 μL of wash buffer is added to 100 μL of RNA extraction buffer (10 mM Tris-HCl pH 7.5, 10 mM MgCl₂, 1% (w/v) SDS) and 100 μL of phenols saturated with water. After 15 minutes of strong stirring with vortex, and then centrifuging for 10 minutes at 12,000 g, the aqueous phase is taken up and set to precipitate at −20° C. for one hour from 2.5 volumes of ethanol in the presence of 0.1 volume of 1M sodium acetate pH 4.8. After centrifugation for 20 minutes at 16,000 g, the pellet is dried in open air and taken up in 10 μL of water.

1.1.9. Fractionation of RNA on a polyacrylamide gel

Fractionation of radioactive tRNA and/or mitochondrial tRNA transcripts is carried out by electrophoresis on a 15% polyacrylamide gel under denaturation conditions. The gel (8×12×0.03 cm) has the following composition: 15% of acrylamide/bisacrylamide (19/1), 7M urea, TBE 1× buffer (90 mM Tris, 2.5 mM EDTA, 90 mM boric acid). Polymerization is obtained by adding 0.07% (w/v) of ammonium persulfate (APS) and 0.035% (v/v) of TEMED. Before deposition, the tRNA transcripts are added with a load buffer volume (95% (v/v) formamide, 20 mM EDTA, 0.05% (w/v) bromophenol blue, 0.05% (w/v) xylene cyanol). The electrophoresis buffer is TBE 1× and migration is carried out under a maximum current of 25 mA. After migration, the nucleic acids are viewed under UV after incubation for 5 minutes in a 0.5% μg/μL ethidium bromide bath. The gel is then incubated for 30 minutes in a solution of 10% (v/v) acetic acid, 20% (v/v) ethanol and then dried for 1 hour in a gel dryer before being exposed against a plate of Phosphorimager (Fuji) and/or subject to autoradiography for viewing the radioactive transcripts.

1.1.10. Fractionation of RNA on a Formaldehyde Agarose Gel

The RNAs of high molecular weight (>500 nucleotides) are separated by electrophoresis on a gel of 1% (w/v) agarose, 18% (v/v) formaldehyde, MOPS 1× (20 mM MOPS, 2 mM sodium acetate, 1 mM EDTA) in a migration buffer MOPS 1×. The RNAs are first denatured for 10 minutes at 70° C. in 4 volumes of load buffer (15 mM MOPS, 60% (v/v) formamide, 15% (v/v) formaldehyde, 4% (v/v) glycerol, 0.025% (w/v) cyanol xylene, 0.025% (w/v) bromophenol blue) and 1 μL of BET (10 μg/μL) and then rapidly cooled for 5 minutes on ice. After 5 minutes of pre-migration of the gel at 70V, the samples are deposited and electrophoresis takes place for 3 hours at 50V. The gel is washed 3 times for 10 minutes in water and twice for 30 minutes in SSC 20× buffer (3M NaCl, 0.3 M trisodium citrate, pH 7). The RNAs are then transferred by capillarity overnight in SSC 20× on a nylon membrane Hybond™-N+ (Amersham). The membrane is rinsed in SSC 2×, set on Whatmann paper impregnated with SSC 2× and fixed for 5 minutes under UV. The membrane is rinsed in water, briefly incubated in a mixture of 0.2% (w/v) methylene blue −0.2 M sodium acetate, at pH 4.8 and then discolored in water. With this step it is possible to view the RNAs and the molecular weight markers which are not labeled radioactively. The membrane is then set for exposure against a Phosphorimager Plate (Fuji) and/or set in autoradiography for viewing the radioactive transcripts.

1.1.11. cRT-PCR (“circularized Reverse Transcription Coupled with a Polymerization Chain Reaction”) Reaction

In a first phase, 1 μg of mitochondrial RNAs are circularized by incubation for 3 hours at 37° C. in the presence of 100 units of T4 RNA ligase (Fermentas) and of 3.4 μM of ATP in a ligation buffer (50 mM Hepes-NaOH pH 8, 10 mM MgCl₂, 10 mM DTT). The enzyme is inactivated for 15 minutes at 65° C. and then the RNAs are precipitated from 2.5 volumes of 100% ethanol in the presence of 0.1 volume of 1M sodium acetate pH 4.8. After centrifugation for 20 minutes at 16,000 g, the pellet is dried in the open air and taken up in 10 μL of water.

The reverse transcription reaction (RT) is then carried out in order to synthesize the complementary DNA (cDNA) corresponding to the ligation between the ends 5′ and 3′ of the analyzed RNA. 400 ng of RNAs are denaturated for 5 minutes at 65° C. in the presence of 20 pmol of specific primer for the sought RNA. The reverse transcription reaction takes place for 1 hour at 52° C. in 20 μL of a reaction medium containing: 100 nmol of each dNTP, 100 nmol of DTT, 4 μL of RT 5× buffer (250 mM Tris-HCl pH 8.3, 35 mM KCL, 15 mM MgCl₂), 100 units of SuperScript III RT™ (Invitrogen) and 40 units of RNAse inhibitor (RNaseOUT™ —Invitrogen). The enzyme is inactivated for 15 minutes at 70° C. One tenth of the products of this reaction is used as template for a PCR reaction.

1.1.12. Microscopic Observations

The microscopic observations were carried out on a laser scanning confocal microscope Zeiss LSM 510. The fluorochrome MitoTracker™ Orange CM-H2TMRos (Molecular Probes) is used for controlling the integrity of the mitochondria. This is 4-chloromethyltetramethylrosamine which is a vital staining agent specifically penetrating into the mitochondria having a membrane potential. It fluoresces in the red (μm: 574 nm) when it is excited at 551 nm. This staining agent is added to the isolated mitochondria at a final concentration of 0.5 pm. UTP Fluorescent Chromatide® Alexa Fluor® 488 (Molecular Probes) used for the labelling of RNA fluoresces in the green (μm: 561 nm) when it is excited at 488 nm. Image processing is accomplished on the software package LSM 510 version 2.8 (Zeiss).

1.1.13. Gel Shift Technique

The RNA-protein interaction was studied by means of the shift gel technique. The procedure used is the procedure No. TB110 of Promega, entitled <<gel shift assay system >>. The reaction is conducted in a 9 μL reaction volume comprising: 0 to 20 μmol of protein, 2 μL of gel shift buffer 5× (20% (v/v) glycerol, 5 mM MgCl₂, 2.5 mM EDTA, 2.5 mM DTT, 250 mM NACl, 50 mM Tris-HCL pH 7.5). The medium is incubated for 10 minutes at room temperature. One μL of radiolabelled tRNA (about 10 fmol corresponding to 20,000 cpm) is then added and the whole is then incubated under mild stirring at 25° C. After migration, the medium is added with one μL of load buffer (250 mM Tris-HCl pH 7.5, 40% (v/v) glycerol). The gel is prepared from a solution of 4% acrylamide/N,N′-methylene bisacrylamide (19/1) in TBE 0.5× buffer (45 mM Tris 45, 1.25 mM EDTA pH 8.3, 45 mM boric acid). The polymerization of the gel is catalyzed by adding 0.04% (w/v) APS and 0.04% (v/v) TEMED. Before charging the samples, the gel is set to a voltage of 350 V for 10 mins. Migration is then carried out for 20 mins at 350 V in the TBE 0.5× buffer. The gel is dried and then exposed against a Phosphorimager plate (Fuji) or subject to autoradiography.

1.2. Results

1.2.1. Interaction between the DHFR protein and the RNA

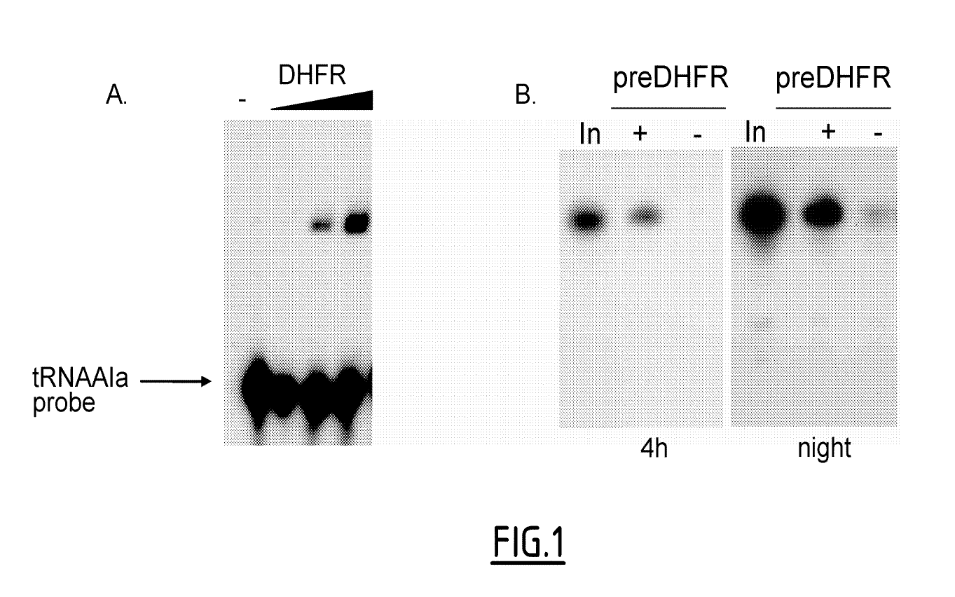

The gel shift technique was used for demonstrating that the recombinant DHFR protein purified by means of the histidine tag is capable of binding nucleic acids. The nucleic acid substrate selected in this case corresponds to the cytosolic tRNAAla of Arabidopsis thaliana (SEQ ID NO: 4). This RNA was obtained by radioactive transcription in vitro from a recombinant plasmid (Carneiro et al., Plant Mol Biol, 26, 1843-1853, 1994). A shift of the tRNAAla transcript in the presence of DHFR was observed (FIG. 1A). The inventors have therefore demonstrated that there exists an interaction between DHFR and nucleic acids.

It should be noted that it has already been demonstrated that human cytosolic DHFR is capable of specifically interacting with a short RNA fragment from its own mRNA (Tai et al., Biochem Biophys Res Comm, 2008). However, it was not known that the DHFR of mice has a much wider interaction spectrum and that it may interact with any nucleic acid.

1.2.2. The pSu9-DHFR Protein is Imported into Isolated Potato Mitochondria

The importing of the fusion protein pSu9-DHFR of 28.6 kDa in isolated potato mitochondria was checked. It was shown that the protein pSu9-DHFR is actually imported into the mitochondria, and that the protein is protected from the action of proteases after import, and cleaved at the targeting sequence for generating a protein of mature size of 22 kDa.

1.2.3. In Vitro Import of the Transcript Corresponding to the TRNAAla in Isolated Potato Mitochondria

It was shown that the import of tRNAAla into mitochondria is strongly enhanced in the presence of the fusion protein pSu9-DHFR (FIG. 1B). Further, it was also demonstrated that with this method the tRNAAla may reach the matrix space of the mitochondria, as demonstrated by the fact that the tRNAAla after import and obtaining mitoplasts, is protected against the action of ribonucleases. The fusion protein pSu9-DHFR may therefore be used for importing nucleic acids into mitochondria.

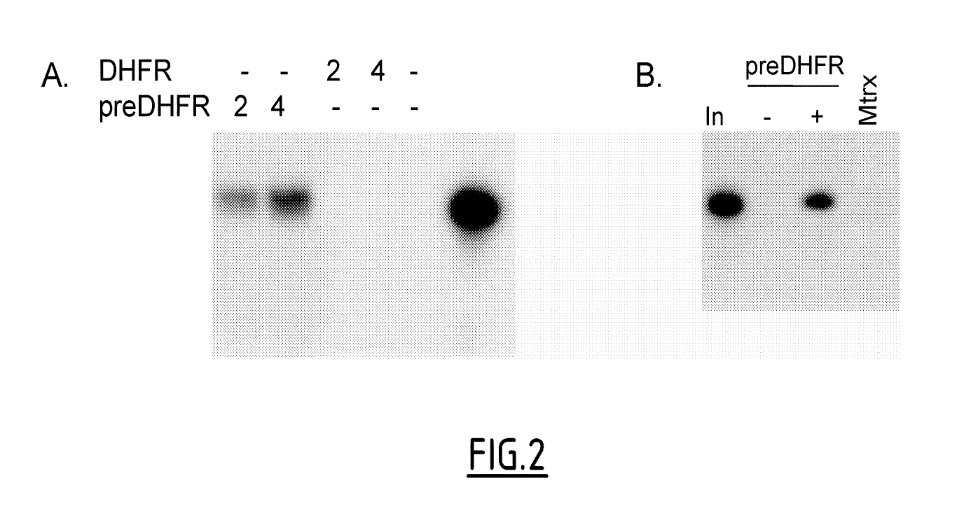

The very strong increase in internalization of the tRNAAla in the presence of the protein pSu9-DHFR in isolated plant mitochondria was corroborated by the following results:
1. Import experiments in the presence of increasing amounts of pSU9-DHFR have shown that the import efficiency for the tRNAAla transcript was correlated with the amount of pSu9-DHFR present in the incubation medium (FIG. 2A).
2. It was found that the recombinant protein DHFR alone (i.e. without any mitochondrial targeting sequence) is unable to carry away tRNAAla into the inside of isolated mitochondria (FIG. 2A).
3. No effect was observed on the import of the tRNAAla transcript in the presence of the fusion protein pSu9-GFP (for <<pSu9—Green Fluorescent Protein >>). The protein pSu9-GFP was obtained in the same way as the protein pSu9-DHFR from a recombinant clone including the mitochondrial targeting sequence pSu9 fused with the sequence coding for GFP (SEQ ID NO: 5), this in the vector pQE40. The protein pSu9-GFP was then overexpressed and purified as mentioned above for the protein pSu9-DHFR. Although the protein pSu9-GFP is properly internalized in vitro in isolated potato mitochondria, this protein is unable to interact with the tRNAAla unlike the protein pSu9-DHFR.
4. It was shown that a substrate analog of the folate, the methotrexate, which is capable of being bound to DHFR (Eilers et Schatz, Nature, 322, 228-232, 1986), inhibits the import of tRNAAla into isolated mitochondria (FIG. 3B).
5. The import of a tRNAAla transcript marked with a fluorophore, the UTP Cromatide® Alexa Fluor® 488, into isolated mitochondria in the presence of pSu9-DHFR was directly viewed by confocal microscopy. The obtained results show that in the absence of pSu9-DHFR, practically no mitochondrion fluoresces while in the presence of the recombinant protein, the quasi-totality of the mitochondria are fluorescent, confirming the presence of tRNAAla at the organelles.

To this day, in vitro import of tRNAAla into isolated potato mitochondria had been observed in the absence of any additional cytosolic factor, but with very low efficiency (Delage et al., Mol Cell Biol, 23, 4000-4012, 2003). In the presence of the recombinant protein pSu9-DHFR, this tRNA is internalized in a much more efficient way in these same mitochondria (from 50 to 100 times, i.e. from 10 to 20% of the RNA put into the presence of the mitochondria at time T=0 of the incubation).

1.2.4. In Vitro Import of the Transcript Corresponding to the Larch Mitochondrial tRNAHis Precursor into Isolated Potato Mitochondria

The importing into isolated potato mitochondria of a transcript of 250 nts corresponding to the precursor form of the larch mitochondrial tRNA(His) (SEQ ID NO: 6) was tested.

In a first phase, two radiolabelled transcripts obtained by transcription in vitro from a recombinant plasmid were used. These transcripts respectively correspond to edited and non-edited forms of larch mitochondrial tRNAHis. Indeed, in plant mitochondria, mitochondrial transcripts undergo an addition process: a certain number of cytidines are converted after transcription into uridines (Gagliardi et Binder, Annual Plant Reviews, 31, 51-96, 2007). The inventors previously showed that larch mitochondrial tRNAHis has three addition sites (Maréchal-Drouard et al., Nucleic Acids Res, 24, 3229-3234, 1996). The internalization of a tRNA precursor in the mitochondrion should lead to its handling by the mitochondrial molecular mechanisms and to its cleavage in 5′ and 3′ by the P and Z RNases respectively. The inventors showed previously that only the edited form of tRNAHis was maturated in vitro. Further, in vivo, the non-edited and non-maturable form is rapidly degraded in the mitochondria (Placido et al., J Biol Chem, 280, 33573-33579, 2005). The obtained results are illustrated in FIG. 3B. After incubation, a migrant RNA with the size of the RNA corresponding to the mature form of tRNAHis is again found in incubated mitochondria in the presence of pSu9-DHFR, and this only when the edited form of the precursor is used.

In a second phase, the non-radiolabelled transcript corresponding to the edited precursor form of the tRNAHis was used for an experiment for import into isolated potato mitochondria and in the presence of pSu9-DHFR. After incubation, the total RNAs were extracted from the mitochondria and maturation of the tRNAHis was analyzed by the cRT-PCR technique. To do this, the complementary primary called P1 (SEQ ID NO: 7) was used for the reverse transcription step and the PCR step was carried out with this same complementary primer and the direct primer, called P2 (SEQ ID NO: 8). The schematic localization of these primers is shown in FIG. 3A. The amplification product was cloned in the vector pGem-T Easy vector (Promega) and the DNA sequence of the clones resulting from this was produced with an Applied Biosystems 3100 apparatus (Perkin Elmer). Fifteen different clones were analyzed. The sequence of the 5′ and 3′ ends of 15 clones is shown in FIG. 3C. These sequences show that all the analyzed molecules, except one, are maturated at their 5′ end. For five of them, the 3′ end is also maturated: the terminal CCA end was added after transcription by the tRNA-nucleotidyl transferase localized in the mitochondrial matrix. This demonstrates that tRNAHis was actually sent to the mitochondrion, since the tRNAHis underwent a maturation process only present inside mitochondria.

To this day, only tRNAs or 5S RNA have been imported into isolated mitochondria of various organisms (Salinas et al., Trends Biochem Sci, 33, 320-329, 2008). The present results demonstrate that nucleic acids of larger size may be imported into mitochondria by the present invention. Considering these results, the fusion protein pSu9-DHFR may be used for internalizing large size RNAs in the mitochondrial matrix space, in an aspecific and desirable way.

1.2.5. In Vitro Import into Isolated Potato Mitochondria of the Transcript Corresponding to the Non-Edited Mitochondrial Form of the Potato mRNA atp9

The import into isolated potato mitochondria was then carried out for the two following transcripts: a transcript of 775 nt (trnH-atp9) corresponding to the precursor form of larch mitochondrial tRNA(His) fused with the non-edited version of potato mitochondrial atp9 (SEQ ID NO: 9) and a transcript of 651 nt (atp9) corresponding to the non-edited version of potato mitochondrial atp9 alone (SEQ ID NO: 10). The chimeric construct with which the transcript trnH-atp9 may be obtained, was obtained by mutagenesis with PCR from the plasmid containing the sequence coding for the edited precursor transcript of larch mitochondrial tRNAHis and from the one containing the sequence coding for the non-edited version of the potato mitochondrial gene atp9. The oligonucleotides required for mutagenesis by PCR are shown as the sequences SEQ ID NOS. 11, 12, 13 and 14. The RNA transcripts corresponding to both of these constructs were synthesized in vitro in either the presence or not of radioactive UTP. These transcripts were then used in tests for in vitro import into isolated potato mitochondria.

It was shown that when the protein pSu9-DHFR is added to the import medium, a much more intense radioactive signal is visible after incubation, both when the RNAs have been extracted from mitochondria and mitoplasts. By estimating each of the radioactive signals relatively to the quantification of the RNAs stained with ethidium bromide and to the specific activity of the transcript, it was possible to estimate that the amount of transcripts imported into the mitochondria was about 3%. No significant difference between the <<mitochondria >> sample and the <<mitoplast >> sample was observed suggesting that these RNAs have reached the matrix space.

In order to reinforce the data obtained above, similar experiments were conducted with non-radioactive transcripts. With RT-PCR experiments it was then possible to analyze their internalization in isolated mitochondria. Both primers used for the RT-PCR analysis have the sequences SEQ ID NOS. 15 and 16. The latter allow discrimination of exogenous RNAs from endogenous RNAs of atp9. Amplification products of the expected size were obtained only during RT-PCR experiments conducted in the presence of reverse transcriptase and from RNAs extracted from mitochondria incubated with each of the transcripts in the presence of pSu9-DHFR. The cloning of these fragments in the vector pGEM-T Easy (Promega) followed by their sequencing gave the possibility of checking that the obtained RT-PCR products actually corresponded to the sequences of the exogenous transcripts. Finally, by analyzing the sequence of these clones, it was possible to demonstrate that some of the <<editable >> sites were actually edited after internalization of the RNA in the isolated mitochondria. The mRNA of the potato mitochondrial gene atp9 has nine edition sites atp9 (Dell'Orto et al., Plant Science, 88, 45-53, 1993).

Among the analyzed sequences, 6.6% of the clones have two of the nine edited sites:

| | |
|---|---|
| 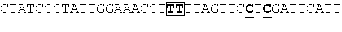 | (sequence of the clones after import); |
| 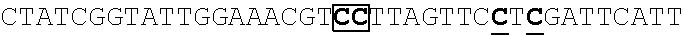 | (non edited sequence). |

The sequences above represent the nucleotides 271 to 309 of the sequence SEQ ID NO: 10. The boxes indicate the edited edition sites, and the underlined nucleotides indicate the non-edited edition sites.

Further, among the analyzed sequences, 23.3% of the clones have two other ones of the nine edited sites:

| | |
|---|---|
| 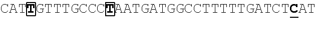 | (sequence of the clones after import); |
| 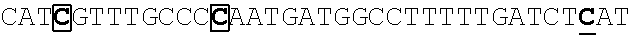 | (non-edited sequence). |

The sequences above represent the nucleotides 388 to 423 of the sequence SEQ ID NO; 10. The boxes indicate the edited edition sites, and the underlined nucleotides indicate the non-edited edition sites.

This demonstrates that the introduced exogenous RNA may attain the enzymes involved in the edition process at the matrix of the mitochondria. Moreover, these results are consistent with the results obtained in vitro demonstrating that the edition sites are not all edited with the same efficiency (Takenaka et al., Mitochondrion, 8, 35-46, 2008).

The whole of the results above show that the pSu9-DHFR protein shuttle system:
Allows internalization in the mitochondria not only of tRNA (75 nt) but also of RNA molecules of a much larger size (775 nt);
allows internalization of RNA independently of a tRNA structure;
allows internalization of these RNAs at the matrix of the mitochondria; and allows targeting in the mitochondria of mRNAs, which are then recognized by the mitochondrial mechanisms involved in gene expression.

### Example 2

### IN Vitro Targeting of the TRNAAla of Plants in Isolated Yeast Mitochondria Via the pSu9-DHFR Protein Shuttle

As this will be shown in Example 2, the shuttle system based on the use of the fusion protein pSu9-DHFR in a plant model may be generalized to any mitochondrion coming from any organism. Indeed, the present invention is partly based on the recognition of the pSu9-DHFR protein by the mitochondrial TOM/TIM complex for import of proteins. As this complex exists in all the mitochondrial systems studied to this day, the shuttle system according to the invention may be generalized to any organism.

2.1. Material and Methods

2.1.1. Purification of Saccharomyces Cerevisae Yeast Mitochondria

The procedure used is the one described by Daum et al. (J Biol Chem, 257, 13075-13080, 1982).

2.1.2. RNA Import in Isolated Yeast Mitochondria

As regards the RNA import experiments in yeast mitochondria, the procedure is the same as the one above described above for potato mitochondria except that the import buffer is inspired from the specific import procedure of the tRNALys (Tarassov et al., J Mol Biol, 245, 315-323, 1995). The 100 μL reaction medium contains 50 μL of yeast 2× import buffer (1.2 mM Sorbitol, 40 mM Hepes-KOH pH 7.4, 2 mM DTT), 4U of creatine phosphokinase, 0.5 μmol of phosphocreatine, 2 mM MgCl₂, 1 mM ATP, 200 μg of mitochondria (protein equivalent), 50 fmol of transcript labeled with [α³²P]UTP (100,000 cpm) and 1 μg of pSu9-DHFR. The RNase mixture and the STOP buffer are made with 1×BB buffer.

In every case, a control experiment conducted in the absence of pSu9-DHFR is carried out. It gives the possibility of checking that the presence of pSu9-DHFR has a positive effect on RNA transport. In order to validate internalization of the RNAs in the matrix space, 100 μL of RNase mixture may also be added after obtaining mitoplasts. In this case, the RNAs protected from the action of RNases cross the internal membrane and are present in the mitochondrial matrix.

2.1.3. Obtaining Mitoplasts

For the yeast, the mitochondria are maintained for 15 minutes in a K1K2 buffer (0.2M K₂HPO₄, 0.8M K₂HPO₄, pH 7.5) before adding a volume of 2× yeast buffer (1.2 M sorbitol, 40 mM Hepes-KOH pH 7.4).

2.2. Results

The transcript corresponding to the plant cytosolic tRNAAla (SEQ ID NO: 4) was tested for its internalization in yeast mitochondria either in the presence or not of the recombinant protein pSu9-DHFR. It was demonstrated that in the presence of the protein pSu9-DHFR, the plant cytosolic tRNAAla which is not normally sent to the yeast mitochondria, is very efficiently internalized in the isolated mitochondria of S. cerevisae yeast. This internalization is accomplished at the level of a matrix since the transcript was found in an equivalent proportion in the mitoplasts.

### Example 3

### In Vitro Addressing of an Oligonucleotide in Potato Or Yeast Isolated Mitochondria Via the pSu9-DHFR Protein Shuttle

The examples above demonstrate that the shuttle system according to the invention allows internalization of allogenic RNAs. It will be demonstrated below that this shuttle system may be used for efficiently internalizing any type of nucleic acid, notably DNA. Firstly, the interaction between a fragment of single strand DNA corresponding to an oligonucleotide of a length of 75 nt and the recombinant protein pSu9-DHFR was validated. Secondly, the internalization of this oligonucleotide in isolated potato or yeast mitochondria was tested.

3.1. Material and Methods

3.1.1. Southwestern Blot Technique

With this technique it is possible to identify proteins interacting with a radiolabelled oligonucleotide. The proteins bound on the Immobilon-P membrane (Millipore) are re-natured overnight at 4° C. in a renaturation buffer (100 mM Tris-HCl pH 7.5, 0.1% (v/v) NP 40). NP-40, an ionic detergent, allows better removal of the SDS. After 4 washes of 15 mins of the membrane in the same buffer at 4° C. with mild stirring, the membrane is saturated for 5 mins at room temperature in a blocking buffer (10 mM Tris-HCl pH 7.5, 5 mM magnesium acetate, 2 mM DTT, 5% (w/v) BSA, 0.01% (v/v) Triton X-100. The filter is then put into the presence of 10⁶ cpm of the radiolabelled oligonucleotide (in the presence of polynucleotide kinase and of [γ-³²P]ATP) in 5 mL of hybridization buffer (10 mM Tris-HCl pH 7.5, 5 mM magnesium acetate, 2 mM DDT, 0.01% (v/v) Triton X-100 and incubated overnight at 4° C. After four 5 min washings at 4° C. with the wash buffer (10 mM Tris-HCl pH 7.5, 5 mM magnesium acetate, 2 mM DDT), the membrane is dried and then subject to autoradiography and/or exposed against a phosphor imager plate for viewing the interaction.

3.1.2. Import of a Radiolabelled Oligonucleotide into Isolated Mitochondria

The 5′ labeling of an oligonucleotide in the presence of [γ³²P] ATP and of polynucleotide kinase is conventionally accomplished according to the recommendations of the supplier (Fermentas). The experiments for importing a radiolabelled oligonucleotide into isolated mitochondria were conducted according to the same procedures as for the import of RNA in potato or yeast mitochondria (see pages above) with one exception: the internalization of DNA in the organelle was validated by adding 100 μL of DNase mixture (25 μg of DNase I, 10 mM MgCl₂, in wash buffer for potato mitochondria and in 1×BB buffer for yeast mitochondria) after obtaining mitochondria or mitoplasts.

3.2. Results

3.2.1. Interaction Between the DHFR Protein and DNA

The Southwestern blot technique was used for demonstrating that the recombinant DHFR protein purified by means of the histidine tag is capable of binding not only RNA but also DNA. The DNA substrate selected in this case corresponds to an oligonucleotide (single strand DNA) corresponding to the sequence of cytosolic tRNAAla of A. thaliana (SEQ ID NO: 4). This chemically synthesized oligonucleotide (Sigma Aldrich) and labeled at 5′ was incubated with a membrane onto which the recombinant proteins DHFR and GFP were transferred. No interaction between GFP and the oligonucleotide was observed while a signal was obtained, reflecting an interaction between DHFR and the oligonucleotide.

3.2.2. In Vitro Internalization of Oligonucleotides in Isolated Potato or Yeast Mitochondria

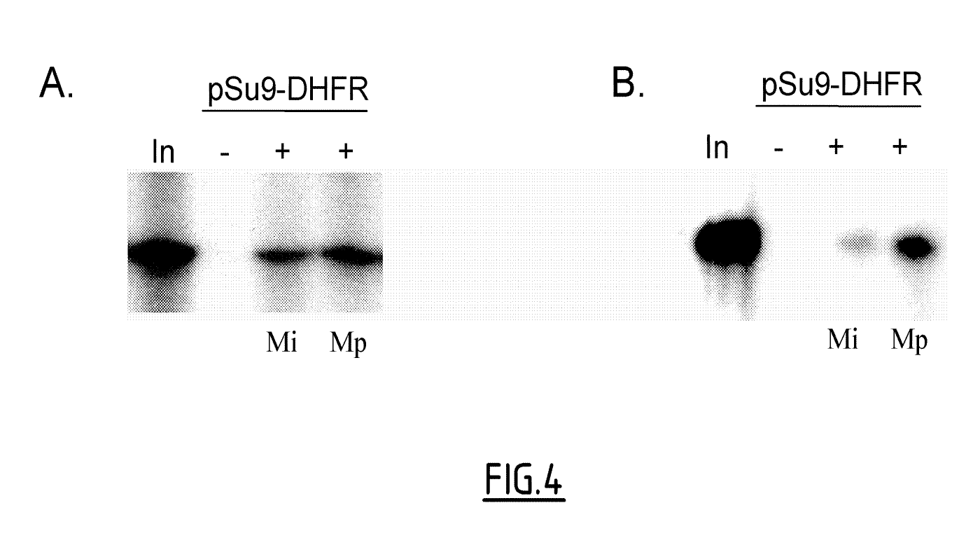

As the interaction between DHFR and the oligonucleotide is possible, the latter was used in in vitro import experiments. The results obtained for the tests carried out with isolated potato and yeast mitochondria are illustrated in FIGS. 4A and 4B respectively. If the oligonucleotide is incubated with mitochondria in the absence of pSu9-DHFR, no signal is visible after autoradiography. On the other hand, the addition of pSu9-DHFR protein into the import medium facilitates the crossing of the oligonucleotide through the double mitochondrial membrane, and a signal is revealed both from extracted fractions of nucleic acids and both from repurified mitochondria and mitoplasts. In this system, the oligonucleotide fraction having reached the matrix space was estimated to be about 1%. This demonstrates that this novel protein shuttle tool may be fully used for transporting DNA into isolated mitochondria.

### Example 4

### Specifically Addressing an RNA Fused with the Stem-Loop Sequence of the Phage MS2 into Isolated Mitochondria Via the Protein Shuttle Formed by the Coat Protein of the Phage MS2 Fused With the Psu9 Mitochondrial Targeting Sequence

The shuttle system according to the invention may be extended to stable genetic systems for specific and controllable introduction of a given nucleic acid into mitochondria. The development of a system for addressing, as desired, a specific RNA, is based on the use of a protein capable of recognizing and of being associated with a specific RNA sequence. In the present example, this protein corresponds to the coat protein of the phage MS2 (also called CP), which is known to recognize and be associated with a specific sequence of RNA adopting a particular stem-loop secondary structure (Beach et al., Curr Biol, 9, 569-578, 1999; Querido et al., Methods in Cell Biol, 85, 273-292, 2008). While the pSu9-DHFR shuttle system allows the import of any RNA, the pSu9-CP shuttle system allows targeted and specific import of an RNA exclusively associated with the stem-loop RNA of the phage MS2.

4.1. Construction of the pSu9-CP Protein

Several Constructs were Made by Mutagenesis Via PCR.

The sequence coding the precursor form of larch mitochondrial tRNAHis was fused on the 3′ side to the sequence coding for the stem-loop portion of the MS2 RNA (SEQ ID NO: 17). This stem-loop sequence is present twice. The primers used for generating this construct by mutagenesis with PCR correspond to the sequences SEQ ID NOS. 20, 21, 22 and 23.

The sequence corresponding to the pSU9 mitochondrial targeting sequence was fused with the protein CP (SEQ ID NO: 18). This construct was cloned in the bacterial expression vector pQE40 and allows the pSu9-CP protein to be obtained. The primers used for generating this construct by mutagenesis by PCR correspond to the sequences SEQ ID NOS. 24, 25, 26 and 27.

4.2. Obtaining the Two Partners and Validation of the Interaction

The substrate RNAs were obtained by in vitro transcription. The pSu9-CP protein was purified by means of the histidine tag present at the C-terminal end of the fusion protein, according to a model similar to the one developed for the recombinant protein pSu9-DHFR.

The interaction between both partners may be validated by the gel shift or Northwestern approaches. The RNA precursor of tRNAHis not fused with the stem-loop RNA portion of MS2 was generated and may be used as a negative control.

4.3. In Vitro Import into Isolated Mitochondria

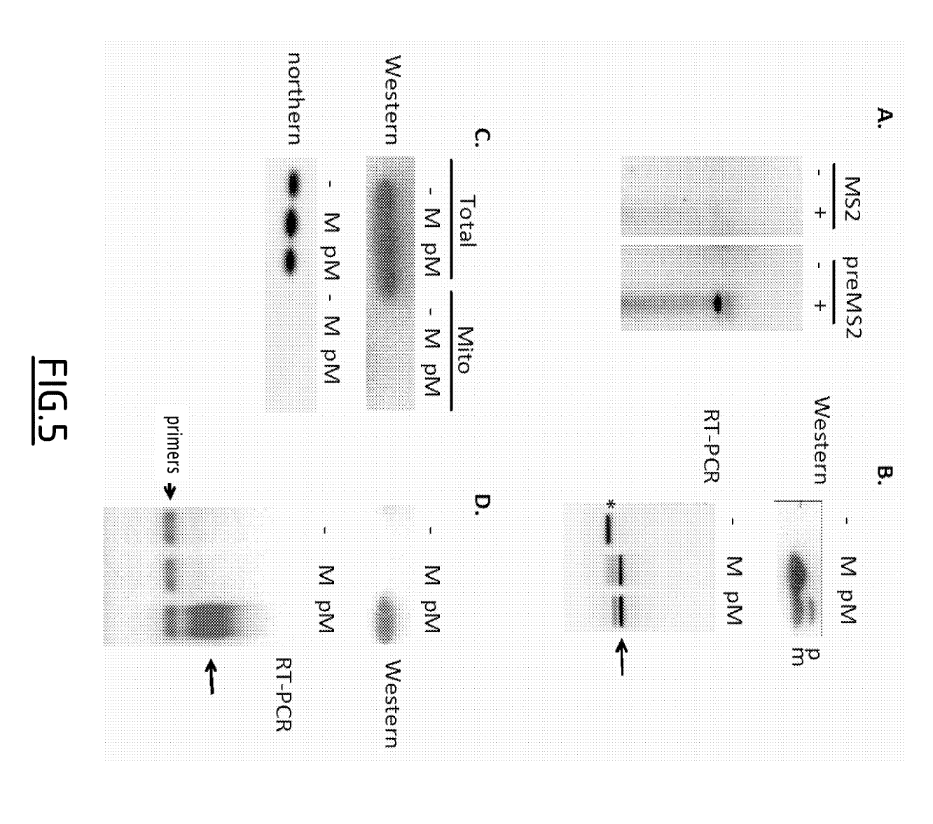

The result obtained for the experiment conducted with isolated potato mitochondria is illustrated in FIG. 5A. When the tRNAHis fused with the sequence coding for the stem-loop portion of the MS2 RNA was incubated with mitochondria in the absence of pSu9-MS2, no signal was visible after autoradiography. On the other hand, the addition of the pSu9-MS2 protein into the import medium facilitated the crossing of the chimeric RNA (MS2 RNAtRNAHis-stem-loop) through the double mitochondrial membrane and a signal was revealed from the extracted fraction of nucleic acids from the mitochondria post-treated with ribonucleases and repurified after import, as described earlier in Example 1 (paragraph 1.1.7). As expected, when the MS2 protein is not fused with the pSu9 mitochondrial targeting sequence (MS2 alone), incorporation of the chimeric RNA into the mitochondria was not facilitated. This shows that the pSu9-MS2 protein shuttle tool may be used for transporting an RNA fused with the stem-loop region which is specific to it into isolated mitochondria.

### Example 5

### Specific Addressing of an RNA Fused with the Stem-Loop Sequence of the Phage MS2 Via the Psu9-MS2 Protein Shuttle Into Mitochondria of Doubly Transformed Yeast Cells

As indicated in the introduction of Example 4, the shuttle system according to the invention may be extended to stable genetic systems for specific and controllable introduction of a nucleic acid into mitochondria. The pSu9-MS2 shuttle system shown in Example 4 and illustrated by the in vitro import of an RNA into isolated mitochondria was extended in the present example to a stable and inducible genetic system in the Saccharomyces cerevisiae yeast.

5.1 Material and Methods

5.1.1 Transformation of Yeast Cells

The pSu9-MS2 construct (SEQ ID NO: 18) was cloned in the vector pESC TRP by means of the cloning cassette MCS2. The second construct corresponding to the antisense sequence of the mitochondrial promoter of the COX1 gene coding for the sub-unit 1 of the yeast mitochondrial cytochrome oxidase (Christianson and Rabinowitz, J. Biol. Chem., 1983, 258:14025-14033) and fused with the sequence coding for the stem-loop portion of the MS2 RNA (SEQ ID NO:35, ATAATGTTATATAAGTAATAATATAATAAAATATCCTAAGGTACCTAATTGCCTAGAAA ACATGAGGATCACCCATGTCTGCAGGTCGACTCTAGAAAACATGAGGATCACCCATG TCTGCAGTATTCCCGGG), was cloned at the cloning cassette MCS1 of the vector pESC TRP. This stem-loop sequence is present twice. This second construct is called anticox-2SL. The primers used for generating this construct by PCR correspond to the sequences SEQ ID NO: 36 (ATAATGTTATATAAGTAATAATATAATAAAATATCCTAAGGTACC) and SEQ ID NO: 37 (CCCGGGAATACTGCAGACATGGG). Both constructs are under the control of a galactose inducible promoter. Further, the pSu9-MS2 construct is cloned fused with a sequence of the vector coding for a tag Myc subsequently allowing the viewing of the expressed protein in the presence of galactose by means of commercially available antibodies and directed against this tag Myc. Competent yeast cells are then transformed by means of this doubly recombinant vector. Similarly a recombinant vector pESC TRP including the same MCS1 construct but exclusively containing the gene coding for the MS2 protein (without the pSu9 targeting sequence) in MCS2 is achieved. It will be used as a negative control. The whole of the procedures from the transformation to the induction of the expression of both partner molecules (the shuttle protein pSu9-MS2 and the anticox-2SL RNA) are described in the manual <<pESCYeastEpitopeTaggingVectors>> provided with the vector by Stratagene.

5.1.2 Purification of Mitochondria and Analysis of the Expression and Import of psu9-MS2 and of anticox-2SL into Mitochondria of Transgenic Yeasts,

The expression of the shuttle protein pSu9-MS2 was analyzed by a Western blot experiment by means of an antibody (Santa Cruz Biotechnology) directed against the tag Myc expressed fused with the protein. To do this, total protein extracts obtained by centrifugation of 20 μL of yeast cells are fractionated on 12% polyacrylamide gel and transferred on an Immobilon P membrane (Millipore). The expression of the anticox-2SL RNA was analyzed by RT-PCR from specific primers (sequences SEQ ID NOS 36 and 37). To do this, total RNA extracts are obtained by the trizol method (Tri Reagent, Molecular Research Center) according to the recommendations of the supplier. The yeast mitochondria are highly purified by having them pass over a saccharose gradient according to the procedure detailed in (Gregg et al. JoVE., which may be consulted on the internet site jove.com/index/details.stp?ID=1417). The quality of the purification of the mitochondria is checked: i) at the protein level by Western Blot experiments with antibodies directed against a mitochondrial protein (protein TOM20) and against a cytosolic protein (protein KAR2), ii) at the RNA level by Northern blot experiments with specific oligonucleotide probes of the trK3 mitochondrial tRNALys (SEQ ID NO: 38, GTGAGAATAGCTGGTGTTG) and of trK2 cytosolic tRNALys(UUU) (SEQ ID NO: 39, GGCTCCTCATAGGGGGCTCG). The trK2 and TrK3 tRNAs are available in (Kolesnikova et al., Human Mol Genet, 13, 2519-2534). To do this, the RNAs are extracted according to the procedure described in 1.1.8 either from the total yeast fraction or from the mitochondrial fraction. The fractionated RNAs on acrylamide gel (1.1.9) are transferred onto a Hybond N Membrane (Amersham). The electrotransfer is carried out in TAE 0.25× buffer (10 mM Tris-Acetate pH 8.0, 0.25 mM EDTA) for 15 minutes under an intensity of 150 mA, and then 30 minutes under an intensity of 500 mA. The membrane is briefly dried and the tRNAs are fixed by UV light irradiation (355 nm) for 3 to 5 minutes. The oligonucleotides are labeled radioactively at their end (3.1.1). The radiolabelled probe is added to the hybridation solution (SSC 6×, 0.5% SDS) in a hybridation roller containing the membrane positioned along the wall. Hybridation is carried out in an oven at a temperature of 45° C. for one night. The membrane is then washed twice for 10 minutes in SSC 2× buffer and then once for 30 minutes in SSC 2× buffer, 0.1% SDS at the hybridation temperature. The membrane is briefly dried and exposed against a Phosphorimager (Fuji) plate or subject to autoradiography. The composition of the SSC 2× is: 30 mM trisodium citrate pH 7.0; 0.3M NaCl. Internalization in the mitochondria of the shuttle protein pSu9-MS2 is checked by a Western blot experiment as described above. Internalization in the mitochondria of anticox-2SL RNA is checked by RT-PCR according to the approach described above.

5.2 Results

FIG. 5B shows that after induction with galactose, these yeast cells transformed with the double construct pSu9-MS2 and anticox-2SL expressed both the shuttle protein in its precursor form (noted p in FIG. 5B) and the RNA, the amplification of which was possible by RT-PCR with specific primers. On the other hand, neither the protein pSu9-MS2, nor the anticox-2SL RNA were present when the yeasts were transformed with the vector pESC TRP alone. Finally, when the vector only included the construct coding for the MS2 protein, the precursor form was not present in the total extract of proteins. It should be noted that in the absence of galactose, no induction either of the protein or of the RNA was obtained. The specific and inducible expression of the constructs was therefore validated. The yeast cells transformed with these three types of constructs were used for preparing mitochondria according to the procedure described above. These mitochondria were free of cytosolic contamination as confirmed by the Western and Northern blot experiments shown in FIG. 5C. The KAR2 protein was present in the 3 total extracts of yeasts transformed with the vector alone (−), with the vector including MS2 and anticox-2SL and with the vector including pSu9-MS2 and anticox-2SL. This protein was not found again in the mitochondrial protein extract (validated by the use of an anti-TOM20 antibody) of the three yeast types. Also, the trK2 cytosolic tRNALys(UUU) was present in the total extracts of nucleic acids but was absent from fractions of mitochondrial nucleic acids (validated by the detection of trK3 mitochondrial tRNALys). Finally, as shown in FIG. 5D, the use of the anti-Myc antibody showed the presence of the protein pSu9-MS2 in its mature form only in the mitochondria of yeast cells doubly transformed with the pSu9-MS2 construct and with the anticox-2SL construct. Also, the presence of anticox-MS2 RNA was only revealed by RT-PCR analysis with specific primers in mitochondria of the same transformed yeast cells. The amplification product obtained by RT-PCR was cloned and sequenced in order to check that it corresponded to the sequence of anticox-2SL RNA. This RNA was not found at the mitochondria of yeast cells transformed with the vector including the construct MS2, demonstrating the importance of the pSu9-MS2 fusion for establishing the stable shuttle system which is the object of the present invention.

As a conclusion, this experiment demonstrates that the pSu9-MS2 (pSu9-CP) protein allows the import of an RNA of interest (in this case the anticox-2SL RNA) fused with the stem-loop portion of MS2 into the mitochondrion of a cell.

1-18. (canceled)

19. A method for in vitro importing a nucleic acid of interest into an isolated mitochondrion comprising the steps of:
contacting a fusion protein between a mitochondrial targeting sequence pSu9 and a protein selected from the DHFR protein and the coat protein of the phage MS2, with the nucleic acid of interest and an isolated mitochondrion, and
leaving them in contact so that the nucleic acid of interest is imported into the isolated mitochondrion.

20. The method according to claim 19, wherein said fusion protein comprises or consists of the pSu9 mitochondrial targeting sequence fused with the DHFR protein.

21. The method according to claim 19, wherein said fusion protein comprises or consists of the pSu9 mitochondrial targeting sequence fused with the coat protein of the phage MS2.

22. The method according to claim 19, wherein:
said pSu9 mitochondrial targeting sequence is coded by a nucleotide sequence at least 80% identical to the sequence SEQ ID NO: 2;
said DHFR protein is coded by a nucleotide sequence at least 80% identical to sequence SEQ ID NO: 1; and
said coat protein of the phage MS2 is coded by a nucleotide sequence at least 80% identical to the nucleotides 208 to 564 of the sequence SEQ ID NO: 18.

23. The method according to claim 19, wherein said nucleic acid of interest is a complete messenger RNA or a complete transfer RNA.

24. A kit for importing a nucleic acid of interest into a mitochondrion comprising:
a) a fusion protein between a pSu9 mitochondrial targeting sequence and a protein selected from the DHFR protein and the coat protein of the phage MS2, or a nucleic acid coding for said fusion protein;
b) at least one reagent for importing the nucleic acid of interest into the mitochondrion; and optionally,
c) instructions for importing the nucleic acid of interest into the mitochondrion.

25. A fusion protein comprising or consisting in a pSu9 mitochondrial targeting sequence fused with the coat protein of the phage MS2.

26. The fusion protein according to claim 25, wherein said pSu9 mitochondrial targeting sequence is coded by a nucleotide sequence at least 80% identical to the sequence SEQ ID NO: 2, and wherein said coat protein of the phage MS2 is coded with a nucleotide sequence at least 80% identical to the nucleotides 208 to 564 of the SEQ ID NO: 18.

27. A nucleic acid coding for the fusion protein according to claims 25.

28. A recombinant vector containing a nucleic acid as defined in claim 27.

29. A combination of nucleic acids comprising:
a) a nucleic acid coding for a fusion protein between a pSu9 mitochondrial targeting sequence and the coat protein of the phage MS2; and
b) a nucleic acid of interest fused with at least one copy of the stem-loop region of the MS2 RNA, or a nucleic acid whose transcription produces this nucleic acid.

30. The combination according to claim 29, wherein the nucleic acids (a) and (b) are comprised by a recombinant vector.

31. A pharmaceutical composition comprising a fusion protein between a mitochondrial targeting sequence pSu9 and a protein selected from the DHFR protein and the coat protein of the phage MS2, and/or a combination according to claim 29.

32. A pharmaceutical composition according to claim 31, wherein said fusion protein comprises or consists of:
the pSu9 mitochondrial targeting sequence fused with the DHFR protein, or
the pSu9 mitochondrial targeting sequence fused with the coat protein of the phage MS2.

33. A pharmaceutical composition according to claim 31, wherein said pSu9 mitochondrial targeting sequence is coded by a nucleotide sequence at least 80% identical to the sequence SEQ ID NO: 2;
said DHFR protein is coded by a nucleotide sequence at least 80% identical to sequence SEQ ID NO: 1; and
said coat protein of the phage MS2 is coded by a nucleotide sequence at least 80% identical to the nucleotides 208 to 564 of the sequence SEQ ID NO: 18.

34. A method for in vitro import of a nucleic acid of interest into a mitochondrion of a cell, comprising the steps:
a) obtaining or preparing a combination of nucleic acids according to claim 29, and
b) introducing said combination of nucleic acids into a cell.

35. A method for obtaining a recombinant plant characterized in that it includes the following steps:
a) obtaining or preparing a combination of nucleic acids according to claim 29;
b) introducing said combination into a plant cell;
c) regenerating an entire plant from the recombinant plant cell obtained in step (b); and
d) selecting the plants having integrated into their genome said nucleic acids.

36. A recombinant plant which may be obtained by the method according to claim 35.

37. Seed or fruit of a homozygous recombinant plant according to claim 36.
